SEQUENCE LISTING (8 sequences)

SEQ ID NO: 1 - DNA, 23 bp, Artificial Sequence
  Genomic target sequence +PAM of Guide RNA
aaaggtggaagacgacccgaagg

SEQ ID NO: 2 - DNA, 23 bp, Artificial Sequence
  Genomic target sequence +PAM of Guide RNA
ctacaccgaccaccaacgcctgg

SEQ ID NO: 3 - DNA, 23 bp, Artificial Sequence
  Genomic target sequence +PAM of Guide RNA
gtacacggaccaccagcggctgg

SEQ ID NO: 4 - DNA, 23 bp, Artificial Sequence
  Genomic target sequence +PAM of Guide RNA
cctgggcctttccgagagacagg

SEQ ID NO: 5 - DNA, 23 bp, Artificial Sequence
  Genomic target sequence +PAM of Guide RNA
taaatgctgtgcggcgccgtcgg

SEQ ID NO: 6 - DNA, 23 bp, Artificial Sequence
  Genomic target sequence +PAM of Guide RNA
gtacctgacgcgcgcccgcaggg

SEQ ID NO: 7 - DNA, 23 bp, Artificial Sequence
  Genomic target sequence +PAM of Guide RNA
gaatccctggaaatcgccgatgg

SEQ ID NO: 8 - DNA, 23 bp, Artificial Sequence
  Genomic target sequence +PAM of Guide RNA
ggtggagattgccgccaccctgg

(19) United States
(12) Patent Application Publication
(10) Pub. No.: US 2024/0293467 A1
(43) Pub. Date: Sep. 5, 2024

(54) DOPAMINERGIC NEURON PROGENITOR CELLS OR CELL DERIVATIVES THEREOF OBTAINED FROM LINEAGE RESTRICTED PLURIPOTENT STEM CELLS

(71) Applicant: Aarhus Universitet, Aarhus C (DK)

(72) Inventors: Mark Denham, Aarhus C (DK); Maimaitili Muyesier, Åbyhøj (DK)

(21) Appl. No.: 18/268,134

(22) PCT Filed: Dec. 20, 2021
(86) PCT No.: PCT/EP2021/086866

§ 371 Date: Jun. 16, 2023

(30) Foreign Application Priority Data
Dec. 21, 2020 (EP) 20215929.9
Sep. 24, 2021 (EP) 21198825.8

(51) Int. Cl. A61K 35/30; A61K 9/00; A61P 25/28; C12N 5/0793

(52) U.S. Cl. CPC A61K 35/30; A61K 9/0085; A61P 25/28; C12N 5/0619; C12N 2506/02; C12N 2510/00

(57) ABSTRACT

The present invention relates to pluripotent stems cells restricted in their capability to differentiate into cell lineages different from Dopaminergic neuron progenitor cells and derivatives thereof by specific gene knockouts. In particular, the present invention relates to dopaminergic neuron progenitor cells or cell derivatives thereof obtained from these lineage-restricted pluripotent stem cells and uses thereof.

### TECHNICAL FIELD OF THE INVENTION

The present invention relates to pluripotent stems cells restricted in their capability to differentiate into cell lineages different from dopaminergic neuron progenitor cells or cell derivatives thereof, by specific gene knockouts. In particular, the present invention relates to dopaminergic neuron progenitor cells or cell derivatives thereof obtained from these lineage restricted pluripotent stem cells and uses thereof.

### BACKGROUND OF THE INVENTION

Midbrain dopaminergic (mDA) neuron development has been an intense area of research during recent years. This is due in part to a growing interest in regenerative medicine and the hope that treatment for diseases affecting mDA neurons, such as Parkinson's disease (PD), might be facilitated by a better understanding of how these neurons are specified, differentiated and maintained in vivo. This knowledge might help to instruct efforts to generate mDA neurons in vitro, which holds promise not only for cell replacement therapy, but also for disease modelling and drug discovery.

Human pluripotent stem cell can be differentiated into all cell types of the body. As such, pluripotent stem cell have been intensely studied as a way to generate specific cell types for cell replacement therapies or for modelling diseases in vitro. An unmet need in the field is, therefore, to be able to reliably and efficiently direct the pluripotent stem cells into the cell type of interest. To date, there are numerous protocols that have been published for differentiating pluripotent stem cells into various cell types; however, none of them are capable of reliably producing a pure population of one cell type.

Mesencephalic dopaminergic (mesDA) neurons develop from the ventral midbrain of the neural tube. The morphogen SHH and members of the WNT family are instrumental in their specification and are essential in establishing the dorsoventral and anterior-posterior (A-P) axis of the embryo respectively (Castelo-Branco et al., 2003; Hynes et al., 1995). For midbrain dopaminergic neurons, high SHH signalling from the notochord is required to specify ventral neural epithelial cells in the neural plate to a floor plate identity, and graded WNT signalling emanating from the posterior regions of the embryo generates posterior neural progenitors. Neural progenitors, in the midbrain region, receive WNT1 and FGF8 from the isthmic organizer, which are involved in patterning the cells to a mesencephalic identity. The isthmic organizer is essential in refining and directing cell identity to the caudal regions of the ventral midbrain and anterior hindbrain. Recapitulating these developmental steps in vitro with human pluripotent stem cells (hPSCs) has been the focus of cell transplantation therapies for Parkinson's disease.

In stem cell differentiation protocols, early and high concentrations of SHH is sufficient to specify neural progenitor cells to a floor plate identity (Fasano et al., 2010). However, for the A-P (also referred to as rostral-caudal) axis, a titrated WNT concentration within a precise range in concentration is required to specify the cells to the caudal midbrain (Kirkeby et al., 2012). Too high a concentration results in hindbrain cell types and a low concentration results in anterior midbrain. Timed delivery of FGF8 has also been shown to improve the specification to the caudal midbrain (Kirkeby et al., 2017). Despite these advances in stem cell differentiation protocols, cell line variability, and the overall yield of mesDA neurons still hampers the use of these cells in the clinic (Nolbrant et al., 2017).

During the complex development, from pluripotency to a mesDA neuron a cascade of transcription factors are expressed that changes as the cell matures, with distinctive transcriptomic profiles defining a specific maturation stage (La Manno et al., 2016).

WO 2016/162747 A1 discloses a method for producing stem cells derived dopaminergic cells for use in treatment of neurodegenerative diseases. The cells are directed towards the desired lineage by plating on a substrate coated with laminin-111/121/521/421 or 511.

Hence, an improved method for producing stem cell-derived dopaminergic cells would be advantageous, and in particular a more efficient and/or reliable method to produce high amounts or more pure populations of stem cell-derived dopaminergic cells would be advantageous.

### SUMMARY OF THE INVENTION

Regulation of transcription factor expression at alternate lineage points can be manipulated to control cell fate choices.

In this study, we chose to exploit the effects of a gene knockout approach as a method to restrict the cell's fate and prevent non-dopaminergic cell lineages, with the aim of enhancing their differentiation to a Mesencephalic dopaminergic (mesDA) fate. Specifically, we looked at the major points when alternate cell fates choices are made at the early developmental stages and explored what transcription factor determinates are critical for those lineages, but were not required for the mesDA fate. By deleting lineage-determinant genes expressed in alternate lineages, we were able to bias the differentiation of the pluripotent stem cells towards a dopaminergic identity. We generated pluripotent stem cells that could be expanded in the pluripotent state; however, when differentiated, it was restricted in its potential. We termed these “lineage-restricted pluripotent stem cells” (LR-PSCs). Specifically, we focused on the A-P axis of the developing neural tube due to the fact that mesencephalic dopaminergic progenitors requires a titrated expression and examined transcription factors that regulate hindbrain and spinal cord cell fates. The anterior hindbrain has been shown to require Gbx2, and the spinal cord is reliant on Cdx family members. By ablating these genes, we increased the percentage of cells adopting a midbrain identity and prevented the specification to a spinal cord fate. Our results show that this approach can be successfully used to robustly generate functional mesDA neurons without a strict requirement for specific concentrations of extrinsic factors. FIGS. 7-10 illustrate how it is possible to generate lineage-restricted stem cells by deleting specific genes from the cells.

In summary, the present invention relates to pluripotent stems cells restricted in their capability to differentiate into cell lineages different from Dopaminergic neuron progenitor cells and derivatives thereof by specific gene knockouts. In particular, the present invention relates to dopaminergic neuron progenitor cells or cell derivatives thereof, obtained from these lineage-restricted pluripotent stem cells and uses thereof.

Thus, an object of the present invention relates to the provision of stem cells, which are restricted in their differentiation potential.

In particular, it is an object of the present invention to provide Mesencephalic dopaminergic (mesDA) neurons, which have medical uses.

Thus, in a broadest aspect of the invention, the invention relates to “lineage restricted” pluripotent stem cells but also dopaminergic neuron progenitor cells or cell derivatives thereof obtained from lineage-restricted pluripotent stem cells and uses thereof. In an embodiment, lineage restriction is obtained by inactivation of one or more master regulator genes in the pluripotent stem cells. Examples of master regulator genes are provided in the different aspects and embodiments of the invention.

Further, inactivation of one or more master regulator genes result in the pluripotent stems cells no longer being capable of differentiating into (or is at least inhibited in differentiating into) as many cell types (different from dopaminergic neuron progenitor cells or cell derivatives thereof) compared to a similar cell type where one or more master regulator genes has not been inactivated.

In sum, cell lines, which have one or more “master regulator” genes inactivated are “lineage restricted”.

Thus, an aspect of the invention relates to a pluripotent stem cell, characterized in that one or more of the genes selected from the group consisting of GBX2, CDX2, CDX1, CDX4, GBX1, HOXA1, HOXA2, HOXB1, HOXB2, HOXA3, NKX6.1, NKX6.2, NKX2.1, NKX2.2, PAX6, BRN3A (POU4F1), PHOX2A, PHOX2B, PITX2, DBX1, and SIM1 are inactivated.

Another aspect of the present invention relates to the use of the pluripotent stem cell according to the present invention for cell lineage restricted cell differentiation of the pluripotent stem cell.

Yet another aspect relates to the use of the pluripotent stem cells according to the present invention for the generation of dopaminergic neuron progenitor cells or cell derivatives thereof.

Yet another aspect of the present invention is to provide a dopaminergic neuron progenitor cell or cell derivatives thereof, differentiated from a pluripotent stem cell according to the invention.

Yet another aspect, the invention relates to a dopaminergic neuron progenitor cell or cell derivatives thereof, characterized in that one or more of the genes selected from the group consisting of GBX2, CDX2, CDX1, CDX4, GBX1, HOXA1, HOXA2, HOXB1, HOXB2, HOXA3, NKX6.1, NKX6.2, NKX2.1, NKX2.2, PAX6, BRN3A (POU4F1), PHOX2A, PHOX2B, PITX2, DBX1, and SIM1 are inactivated.

Still another aspect of the present invention is to provide dopaminergic neuron progenitor cell or cell derivatives thereof according to the invention, for use as a medicament, such as in stem cell therapy.

In another aspect, the invention relates to the dopaminergic neuron progenitor cell or cell derivatives thereof according to the invention, for use in the treatment, prevention or alleviation of neurodegenerative disorders.

In an aspect, the invention relates to the use of the dopaminergic neuron progenitor cell or cell derivatives thereof according any to the invention in drug screenings assays.

Yet an aspect of the invention relates to a method for providing a pluripotent stem cell according to the invention, the method comprising inactivating at least one gene selected from the group consisting of GBX2, CDX2, CDX1, CDX4, GBX1, HOXA1, HOXA2, HOXB1, HOXB2, HOXA3, NKX6.1, NKX6.2, NKX2.1, NKX2.2, PAX6, BRN3A (POU4F1), PHOX2A, PHOX2B, PITX2, DBX1, and SIM1 in a pluripotent stem cell.

Yet a further aspect of the invention relates to a method for providing dopaminergic neuron progenitor cells or cell derivatives thereof the method comprising
providing a pluripotent stem cell according to the invention; and
differentiating the provided pluripotent stem cell into dopaminergic neuron progenitor cells or cell derivatives thereof.

Advantages of the cells according to the invention are at least:
Restricted differentiation increases the amount of correct (neuronal) cells (see e.g. examples 2-5).
Restricted differentiation limits the risk of dangerous undesired differentiation after grafting in a patient.
Restricted differentiation allows the correct neuronal cell type to be obtained even when a sub-optimal concentration of growth factors or small molecules are used or when they are absent (see e.g. examples 3-4).
Restricted differentiation allows development of electrophysiologically mature neurons (see e.g. example 8).
Restricted differentiation allows robust population of mesDA neurons in an in vivo Parkinson's rat model resulting in rapid motor recovery (see e.g. example 9).

### BRIEF DESCRIPTION OF THE FIGURES

FIG. 1

Differentiation to day 4 caudal neural progenitors. A) Schematic diagram of the day-4 CNP differentiation protocol. B) QPCR analysis of OTX2 in H9, GBX2 and 4xKO conditions at day 4 CNPs. C) QPCR analysis of CDX2 in H9, GBX2 and 4xKO conditions. D) Heatmap of pluripotent genes and neural genes that represent the anterior, midbrain, hindbrain and spinal cord regions in H9 GBX2 and 4xKO lines at day-4 CNPs. E) Schematic diagram of generation of 4xKO cell line including single cell clonal selection of CRISPR modified hESCs. F) Generation of 8xKO knockout cell line from the 4xKO cell line. The 4XKO cell line was transduced with lentivirus containing guide strands that target GBX1, HOXA1, HOXA2 and HOXB1.

FIG. 2

Differentiation to day 11 caudal neural progenitors. A-P) RNA expression analysis of; midbrain genes OTX2, PAX2, PAX5, PAX8 and EN1; hindbrain genes IRX3, HOXA2, HOXB2, HOXB1, KROX20, MAFB, HOXA3, HOXA4; and spinal cord gene HOXC6, HOXB8 and HOXC10. Q) Schematic diagram of the day-11 CNP differentiation protocol.

FIG. 3

Differentiation to ventral midbrain progenitors. A) Schematic diagram of the caudal midbrain differentiation protocol. B-G) Expression of OTX2, LMX1A, FOXA2, EN1, CNPY1 and HOXA2 in H9 and 4xKO cells at day-16 across the range of GSK3i (CHIR99021) concentrations.

FIG. 4

Single-cell sequencing of 1 μM neural progenitors. Examination of genes expressed along the A-P axis in H9 and 4xKO cells at day 16 of midbrain differentiation using 1 μM of GSK3 (CHIR99021).

FIG. 5

Generation of 8xKO cell line. A) The day-4 CNP differentiation protocol. B) OTX2 expression in H9, 4xKO, 8xKO in day-4 CNP differentiated under 3 μM of GSK3i. C) Percentage of cell expressing OTX2 in day-4 CNP differentiated under 3 μM of GSK3i. D-E) Heatmap showing log 2 centered expression of selected genes across H9 hESCs, H9 at day 4, 4xKO at day 4 and 8xKO at day 4.

FIG. 6

A: Single-cell analysis of the 4xKO cell line compared to control (H9) cell line at day-16 of a dopaminergic neuron differentiation protocol.

Percentage of cells that express genes that represent cell types along the dorsal ventral axis are shown. FOXA2 is expressed in cells of the floor plate. Lateral floor plate and basal plate are indicated by the expression of either NKX6.1, NKX6.2, NKX2.2, PHOX2A and PHOX2B.

B: Single-cell analysis of the 4xKO cell line and control (H9) cell line at day-28 of a dopaminergic neuron differentiation protocol.

Single-cell analysis at day 28 of dopaminergic differentiation, in 4X and H9 cells combined, identified 15 cell clusters. Expression of the anterior ventral marker NKX2.1 and the medial marker for the dorsal ventral axis PAX6 was investigated.

FIG. 7

Schematic overview showing differentiation of hESCs into Dopaminergic Neurons. Examples of cell types and extrinsic factors such as growth factors, which control cell fate are indicated.

FIG. 8

Schematic overview showing differentiation of hESCs into Dopaminergic Neurons. In comparison to FIG. 7, examples of transcription factors controlling cell fate is also included.

FIG. 9

Schematic overview showing differentiation of hESCs into Dopaminergic Neurons. In comparison to FIG. 8, it is exemplified how deleting specific transcription factors blocks certain cell fates.

FIG. 10

Schematic overview showing differentiation of hESCs into Dopaminergic Neurons. In comparison to FIG. 9, it is exemplified how deleting additional specific transcription factors an even further restricted differentiation potential is achieved.

FIG. 11

Single-cell analysis at 62 DIV. A) Heatmap of the expression of selected genes illustrating the identity of the clusters. B) Violin plot of LMX1A, TH, FOXA2 and EN1 in 62 DIV clusters. C) Violin plot of PDLIM1 and SEMA3C in 62 Div clusters. D) Violin plot of NR4A2, EN1, KCNJ6, CALB1 and FOXA2 in TH sub-clusters at 62 DIV.

FIG. 12

Generation of functional ventral midbrain DA neurons in vitro. A)

Schematic diagram of the long-term (62 DIV) neuronal differentialtion protocol. B) Representative response (top trace) to a depolarizing current injection (bottom trace) showing firing of repetitive action potentials. C) Example of spontaneous firing at a resting membrane potential of −45 mV showing burst-like events. Overshooting spikes occurred in groups interspersed by periods of subthreshold membrane oscillation. D) Frequency distribution of spontaneous cell firing showing firing frequencies ranging between 1 and 5 Hz (n=16 cells). E) Dopamine content (normatilized to protein concentration) in 4X and H9 cells at 79 DIV, as measured by HPLC. The data are presented as the mean±SD; n=3. An unpaired t-test was used to compare groups. **P<0.01.

FIG. 13

In vivo analysis of cells transplanted into a Parkinson's disease rat model. A) Overview of the in vivo study. Unilateral 6-OHDA-induced MFB lesions were generated (week-4) and confirmed 3 weeks later by the cylinder and amphetamine-induced rotation tests. The animals were subdivided into 3 groups with similar average scores on the rotation test. Four weeks after lesioning (week 0), two of these subgroups were transplanted with 250,000 cells (H9 or 4X cells), and the third group did not undergo transplantation (6-OHDA lesion group). The rotation test was repeated at weeks 8 and 18 posttransplant, and the cylinder test was repeated at week 18. The animals were killed at week 19 posttransplantation (23 week after lesioning) for histological analysis. B-C) Amphetamine-induced rotation (B) and cylinder tests (C) of 6-OHDA-lesioned rats 3-weeks post lesion, showing comparable behavior among 6-OHDA, H9 and 4X groups, subdivided prior to transplantation. For B-C), N=8 for 6-OHDA, N=9 for H9, N=10 for 4X. D) Amphetamine-induced rotational asymmetry. Two-way repeated measures ANOVA followed by Sidak's multiple comparison test; time: F (1.689, 35.46)=19.50, P<0.0001; treatment: F (2, 21)=15.23 P<0.0001. **P<0.01 and ****P<0.0001 vs. the 4X cell-transplanted group at the same time point. §§ P<0.01 and ssssp<0.0001 vs. the same group at week-1. E) The use of each forelimb (contra or ipsi) and both forelimbs in the cylinder test was analyzed by two-way repeated measures ANOVA followed by Sidak's multiple comparison test with time and group as variables. Time×group: both: F (2, 22)=5.785, P=0.009; ipsi: F (2, 22)=8.800, P=0.001; contra: F (2, 22)=4.642, P=0.021. *P<0.05 and **P<0.01 vs. the same group at −1 week. $P<0.05 and $$P<0.01 vs. the 6-OHDA lesion group at the same time point. £P<0.05 and ££P<0.01 vs. the H9 cell-transplanted group at the same time point. The data in D) and E) are presented as the mean±SEM. n=7 rats in the 6-OHDA lesion group, n=9 rats in the 4×cell-transplanted group, and n=8 rats in the H9 cell-transplanted group). F) Representative photos of coronal sections from all three groups immunostained for TH. Higher magnification images of the areas in the frame are shown on the right. Scale bars, 50 μm for all three photos in the column. G) shows the estimated numbers of TH-positive cells in the grafts. H) shows the yield of TH-positive neurons per 100,000 grafted cells. I) shows the volume of the TH-positive graft. J) Quantitative analysis of the immunofluorescence data showing the percentages of GIRK2/TH and CALB1/TH double-positive cells within 4X cell grafts. The data are presented as the mean percentage+SD (n=9 rats).

The present invention will now be described in more detail in the following.

### DETAILED DESCRIPTION OF THE INVENTION

### Definitions

Prior to discussing the present invention in further details, the following terms and conventions will first be defined:

### Dopaminergic Neuron Progenitor Cells

In the present context, “dopaminergic neuron progenitor cells” is to be understood as cells including mesencephalic progenitor dopaminergic neurons, caudal midbrain progenitors, midbrain floor plate progenitors, etc, which can develop into dopaminergic neuron cells.

The dopaminergic neuron cells may be a cell population containing other types of cells. The cell population is preferably a cell population, which does not contain a serotonin neural cell. The dopaminergic neuron cells are preferably a cell population containing FOXA2, LMX1A, EN1 and TH positive cells.

In an embodiment, the dopaminergic neuron progenitor cells are preferably a cell population containing FOXA2, LMX1A, OTX2, EN1, SPRY1, WNT1, CNPY1, PAX8, ETV5, PAX5, SP5, and/or TLE4 positive cells.

### Derivatives of Dopaminergic Neuron Progenitor Cells

In the present context “Derivatives of Dopaminergic Neuron progenitor Cells” is to be understood as cells which the “dopaminergic neuron progenitor cells” can develop into, such as dopaminergic neuron cells, mesencephalic dopaminergic neurons, etc.

### Mesencephalic Dopaminergic Neuron

In the present context the term “Mesencephalic dopaminergic neuron”, refers to the cells being from the midbrain region of the developing brain. Thus, mesencephalic dopaminergic neurons are a subset of dopaminergic neurons.

### Lineage-Restricted

In the present context, the term “lineage-restricted” refers to cells, such as pluripotent stem cells, which is no longer capable of differentiating into (or is at least inhibited in differentiating into) as many cell types compared to a similar cell type which has not been “lineage-restricted”.

In present context, “lineage-restriction” is achieved by inactivating (such as by gene knockout) one or more genes in the cell line as defined in the claims.

### Stem Cells

A stem cell is an undifferentiated cell from which specialized cells are subsequently derived.

The pluripotent stem cells which may be used in the present invention are stem cells having pluripotency which enables the cells to differentiate into any cells existing in the living body, which pluripotent stem cells also have growth ability. Examples of the pluripotent stem cells include, but are not limited to, embryonic stem (ES) cells, embryonic stem cells derived from a cloned embryo obtained by nuclear transfer (“ntES cells”), germline stem cells (“GS cells”), embryonic germ cells (“EG cells”), induced pluripotent stem (iPS) cells, and pluripotent cells derived from cultured fibroblasts and bone marrow stem cells (Muse cells). The pluripotent stem cells are preferably ES cells, ntES cells or iPS cells.

Pluripotent stem cells as used in the present invention are generated with the proviso that no human being is cloned and no human embryo is used for industrial or commercial purposes during the process.

Embryonic stem cells possess extensive self-renewal capacity and pluripotency with the potential to differentiate into cells of all three germ layers. They are useful for therapeutic purposes and may provide unlimited sources of cells for tissue replacement therapies, drug screening, functional genomics and proteomics.

Induced pluripotent stem cells (also known as iPS cells or iPSCs) are a type of pluripotent stem cell that can be generated directly from a somatic cell. It is noted that in an alternative aspect of the present invention the pluripotent stem cell is considered also to include astrocytes or reprogrammed/reprogrammable astrocytes. It is well-known that astrocytes can be reprogrammed to differentiate in to other cell types such as neurons.

### Master Regulator

In genetics, a “master regulator” is a gene at the top of a gene regulation hierarchy, particularly in regulatory pathways related to cell fate and differentiation. A master regulator gene could also be called a “cell identity determinant” or “cell type determinant”. Thus, the genes which could be inactivated according to the present invention could be considered encompassing master regulator genes.

### Neurodegenerative Disease

Degenerative nerve diseases affect many of the body's activities, such as balance, movement, talking, breathing, and heart function. Many of these diseases are genetic. Sometimes the cause is a medical condition such as alcoholism, a tumor, or a stroke. Other causes may include toxins, chemicals, and viruses. Sometimes the cause is unknown.

In the present context, the term “Neurodegenerative disease” includes Alzheimer's disease, Amyotrophic lateral sclerosis, Friedreich's ataxia, Huntington's disease, Lewy body disease, Parkinson's disease and Spinal muscular atrophy.

Neurodegeneration is the progressive loss of structure or function of neurons, including death of neurons. Many neurodegenerative diseases-including amyotrophic lateral sclerosis, Parkinson's disease, Alzheimer's disease, Huntington's disease, and prion diseases-occur as a result of neurodegenerative processes.

Parkinson's disease (PD) is a neurodegenerative disease attributed to the loss of midbrain dopaminergic (DA) neurons. Parkinson's disease is a particularly interesting target for stem cell based therapies due to the relatively focal degeneration of a specific type of mesencephalic dopamine (mesDA) neuron. Proof-of-concept that cell replacement therapy for Parkinson's disease has been obtained in a number of clinical trials. Parkinson's disease (PD) is a neurodegenerative disease attributed to the loss of midbrain dopaminergic (DA) neurons.

In a preferred embodiment the generated dopaminergic neuron progenitor cells according to the present invention is for use in the treatment or alleviation of Parkinson's disease.

### Lineage Restricted Pluripotent Stem Cells

As outlined above, part of the present invention relates to pluripotent stems cell(s) restricted in their capability to differentiate into cell lineages different from Dopaminergic neuron progenitor cells and derivatives thereof by specific gene knock-outs. In particular, the present invention relates to dopaminergic neuron progenitor cells or cell derivatives thereof obtained from these lineage-restricted pluripotent stem cells and uses thereof. Thus, an aspect of the invention relates to a pluripotent stem cell, characterized in that one or more of the genes selected from the group consisting of GBX2, CDX2, CDX1, CDX4, GBX1, HOXA1, HOXA2, HOXB1, HOXB2, HOXA3, NKX6.1, NKX6.2, NKX2.1, NKX2.2, PAX6, BRN3A (POU4F1), PHOX2A, PHOX2B, PITX2, DBX1, and SIM1 are inactivated.

In a preferred embodiment, one or more of genes selected from the group consisting of GBX2, CDX2, CDX1, CDX4, GBX1, HOXA1, HOXA2 and HOXB1 are inactivated. As outlined in the examples section, cell lines have been produced, with 1, 4 or 8 genes knocked-out. These cell types were restricted in differentiating into other lineages than Dopaminergic neuron progenitor cells and derivatives thereof.

In an embodiment, both alleles of one or more of the listed genes are inactivated. It is considered most efficient if both alleles are inactivated.

In yet an embodiment, at least two of the genes selected from the group consisting of GBX2, CDX2, CDX1, CDX4, GBX1, HOXA1, HOXA2, HOXB1 and HOXB2 are inactivated, such as at least three genes, preferably such as at least four genes, such as at least five genes, such as at least six genes, such as at least 7 genes, or more preferably such as at least eight genes are inactivated.

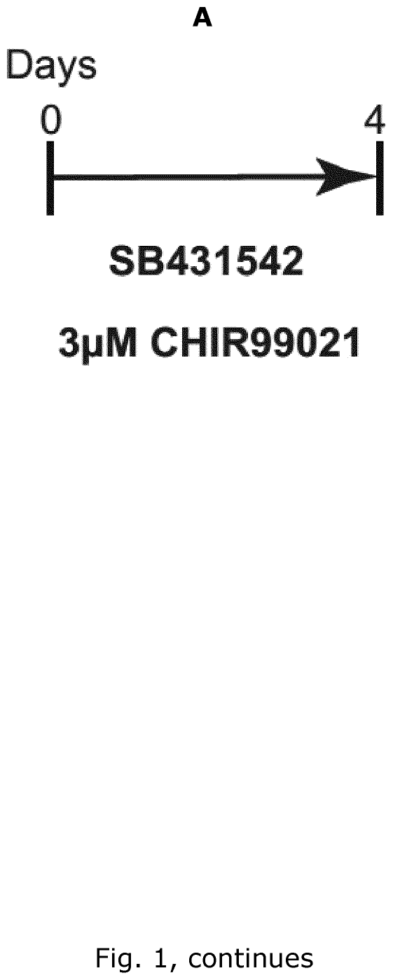

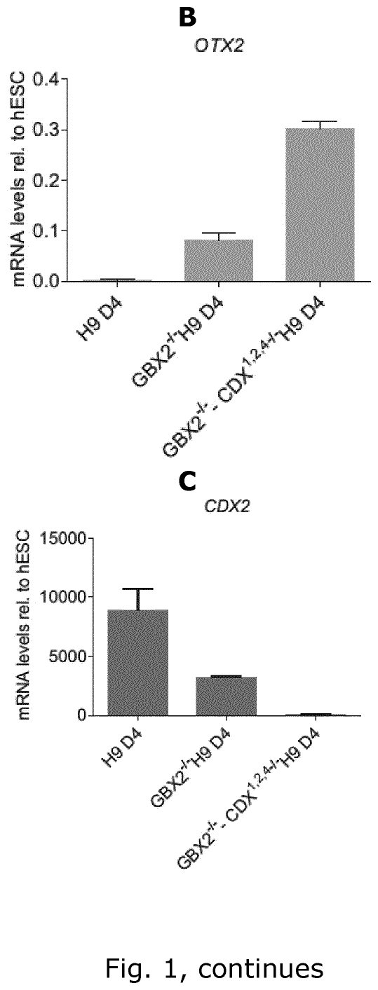

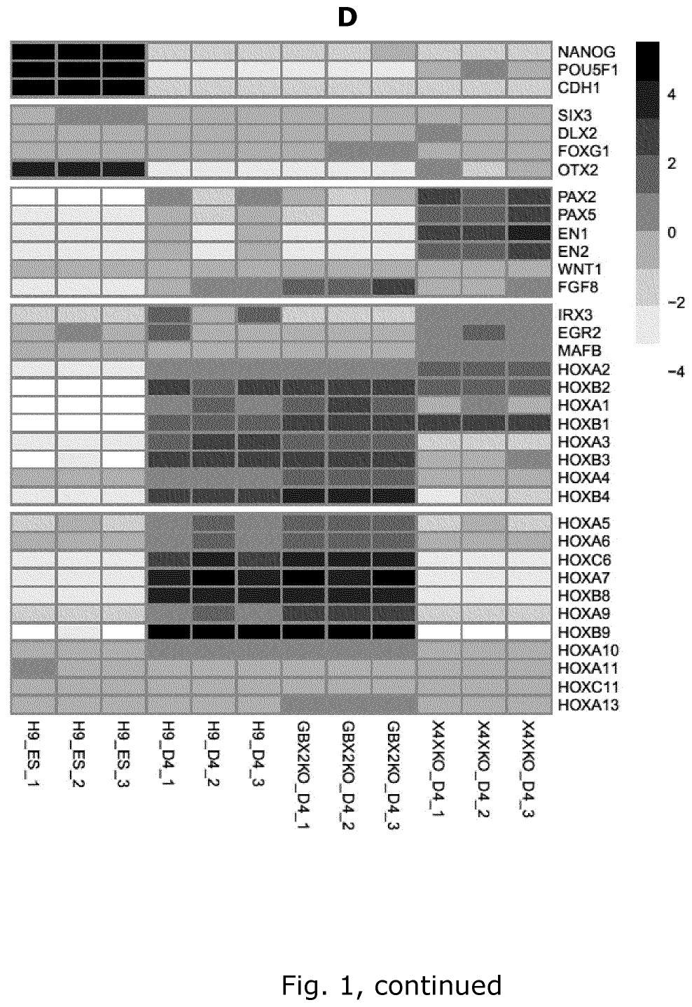

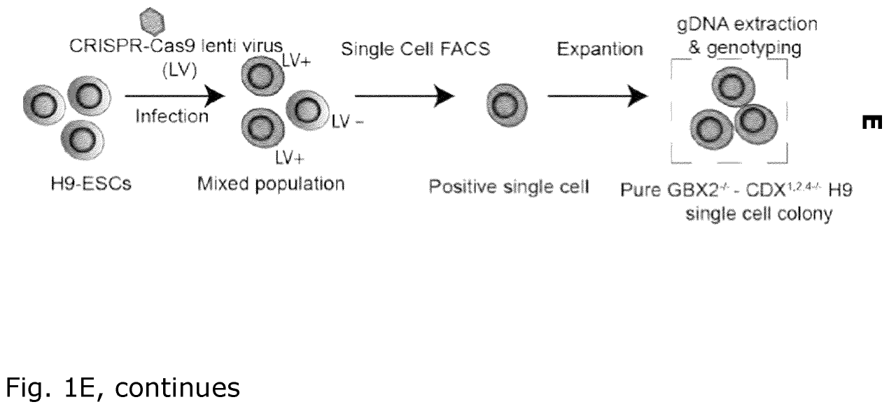

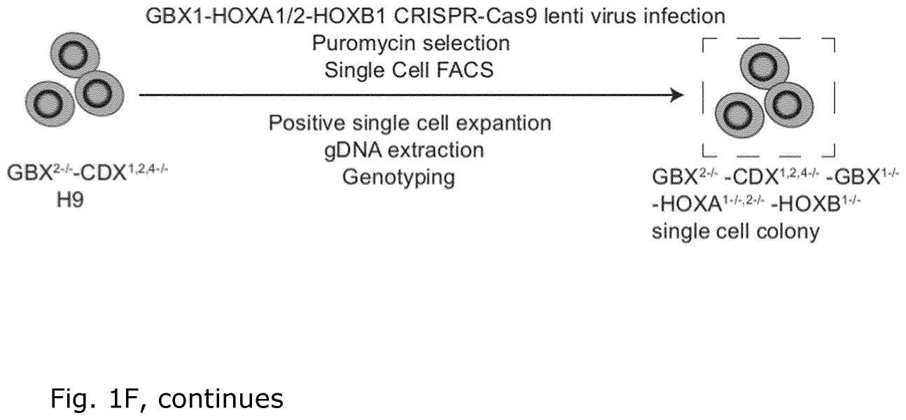

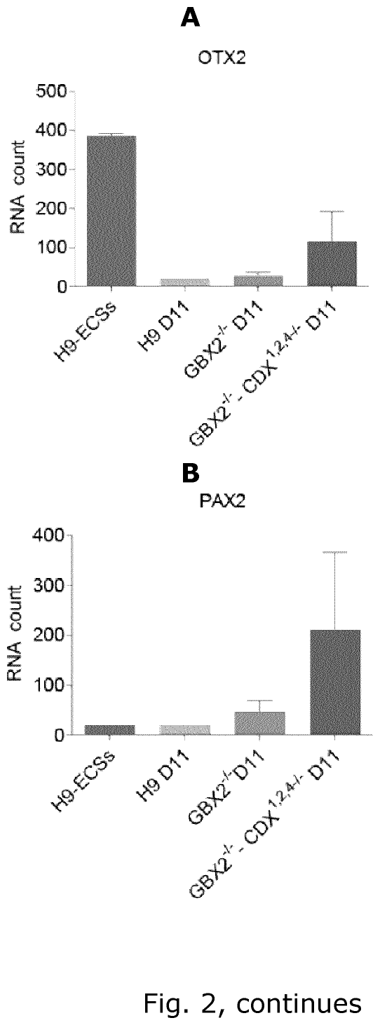

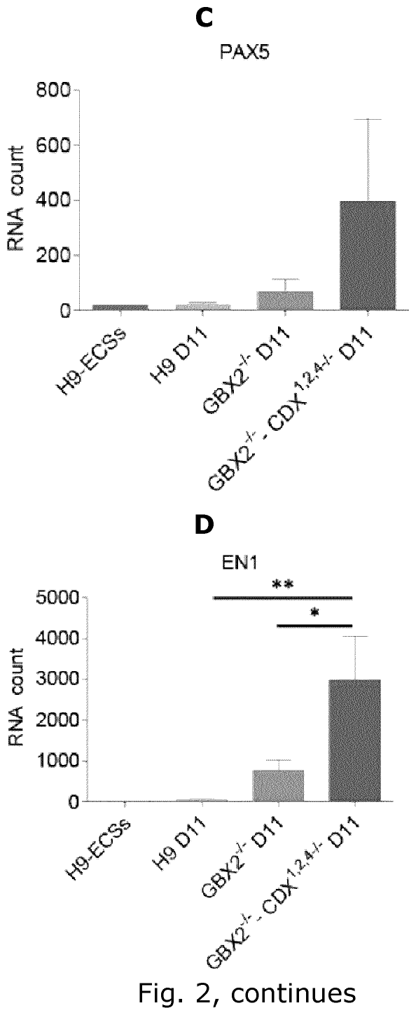

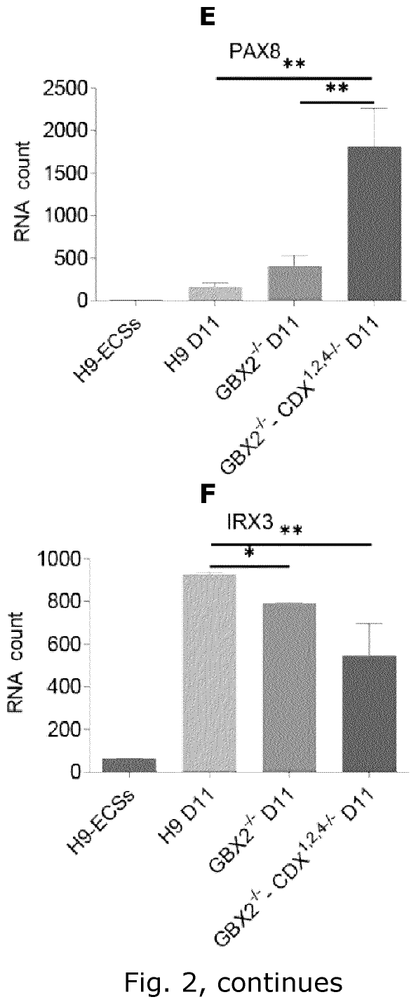

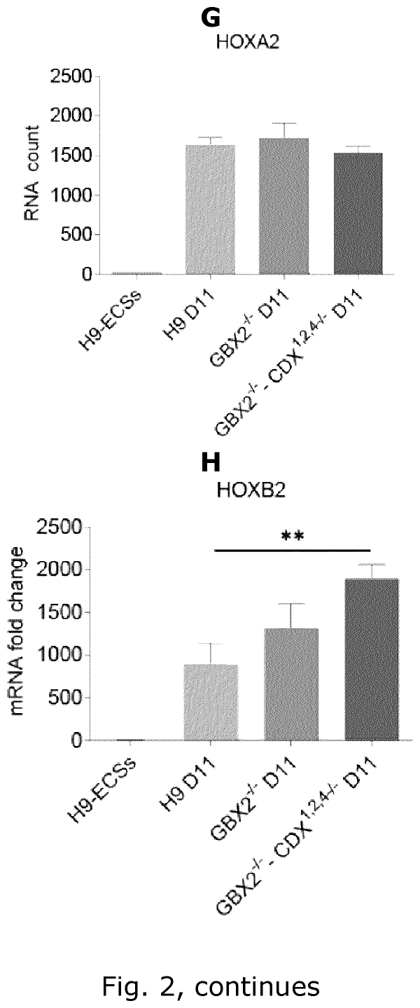

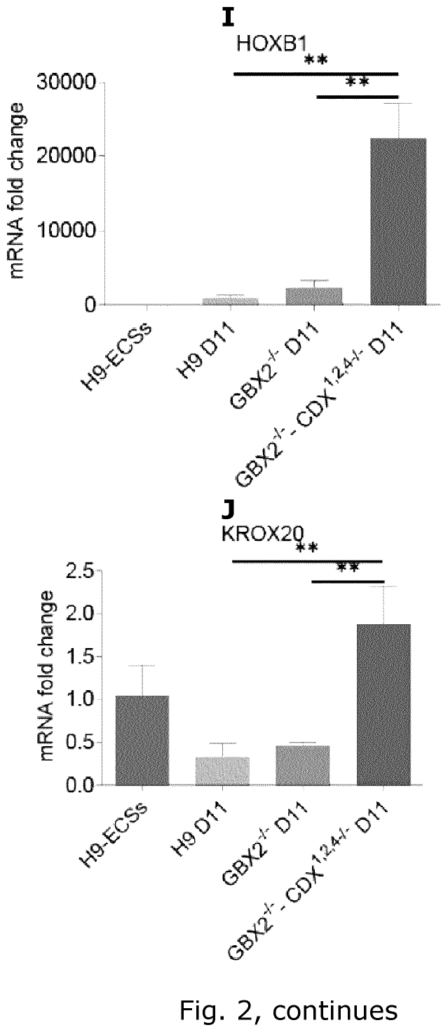

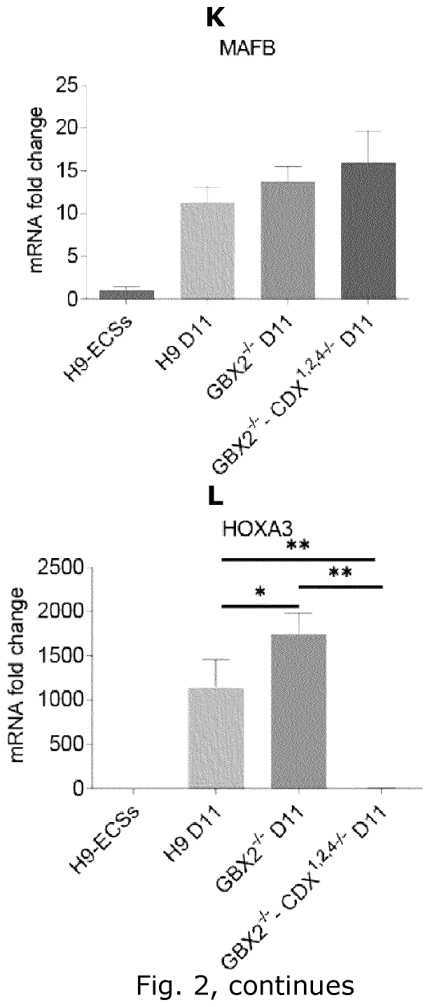

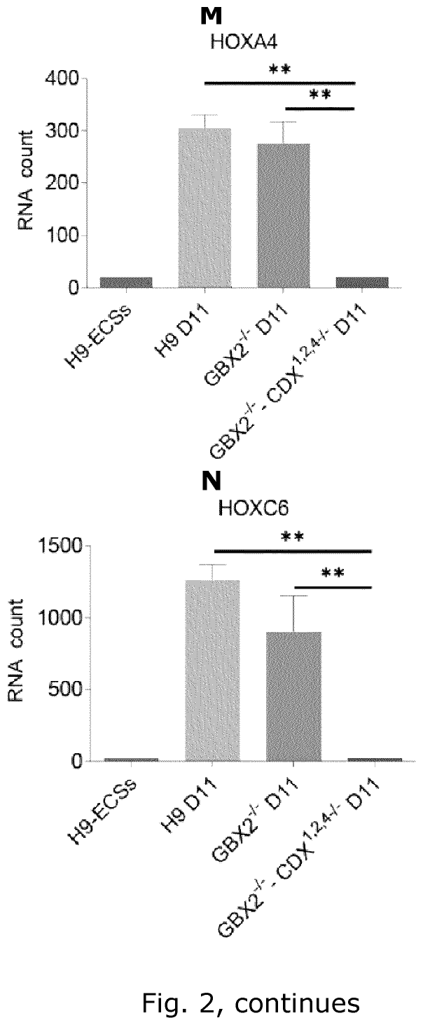

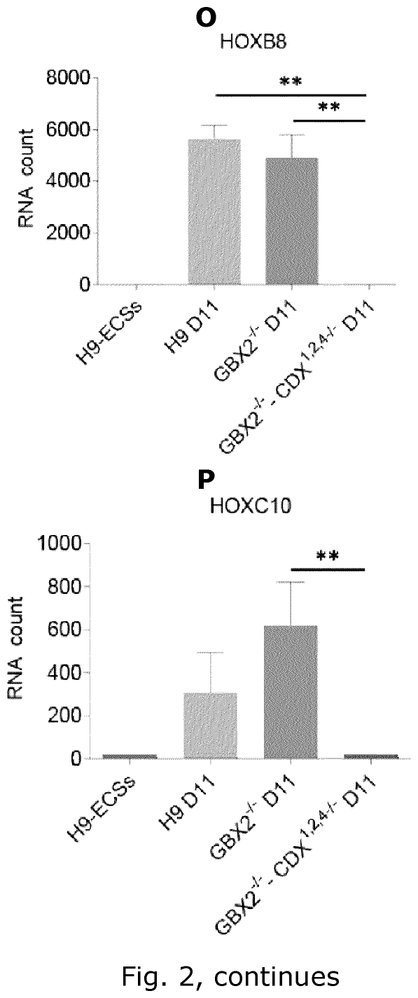

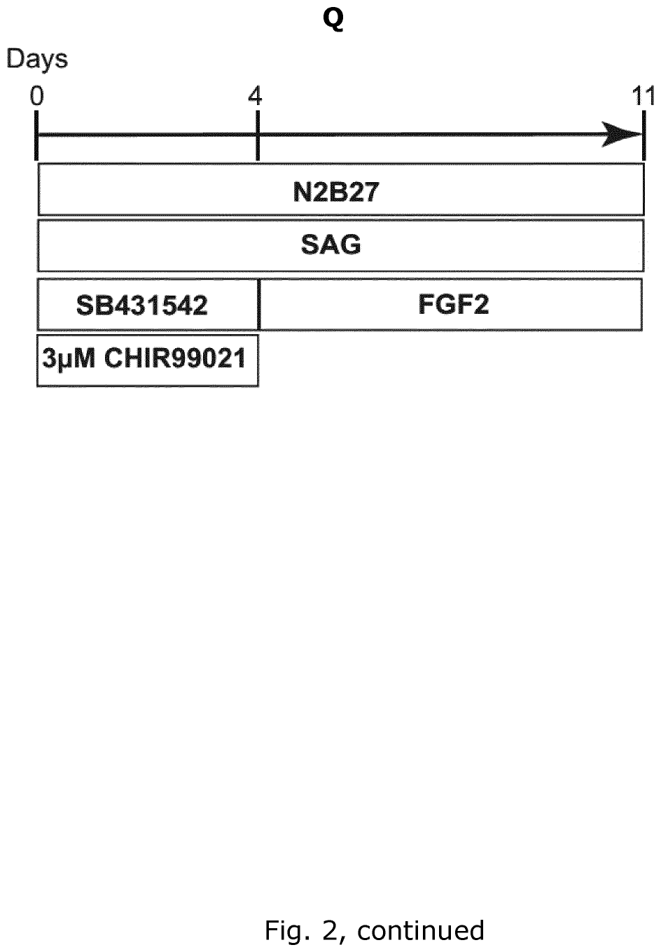

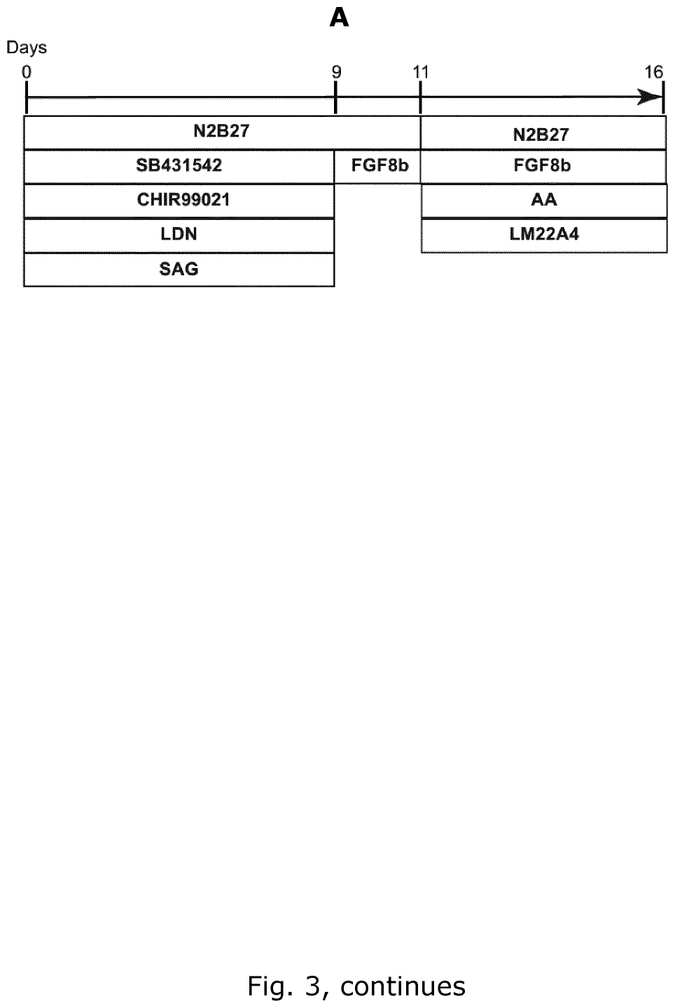

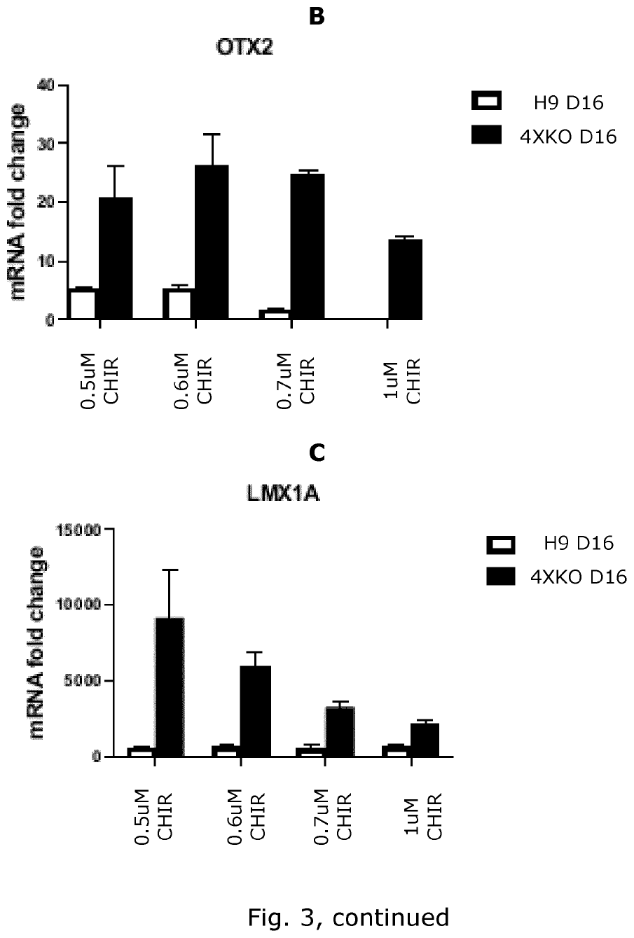

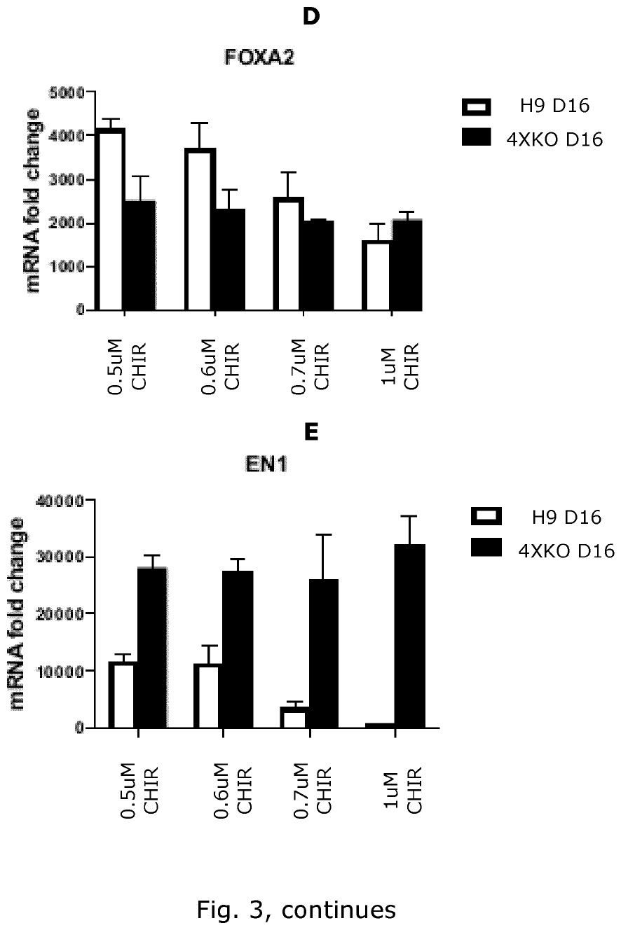

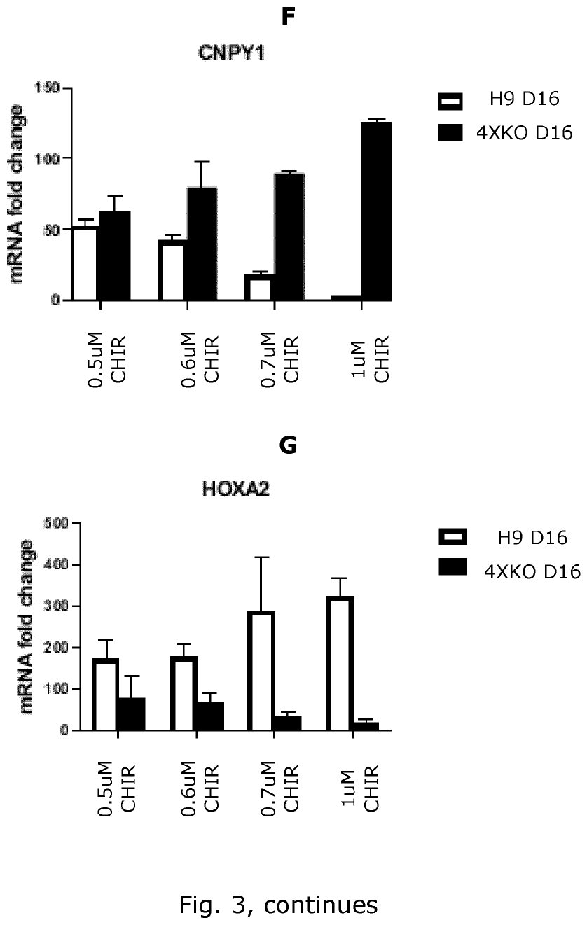

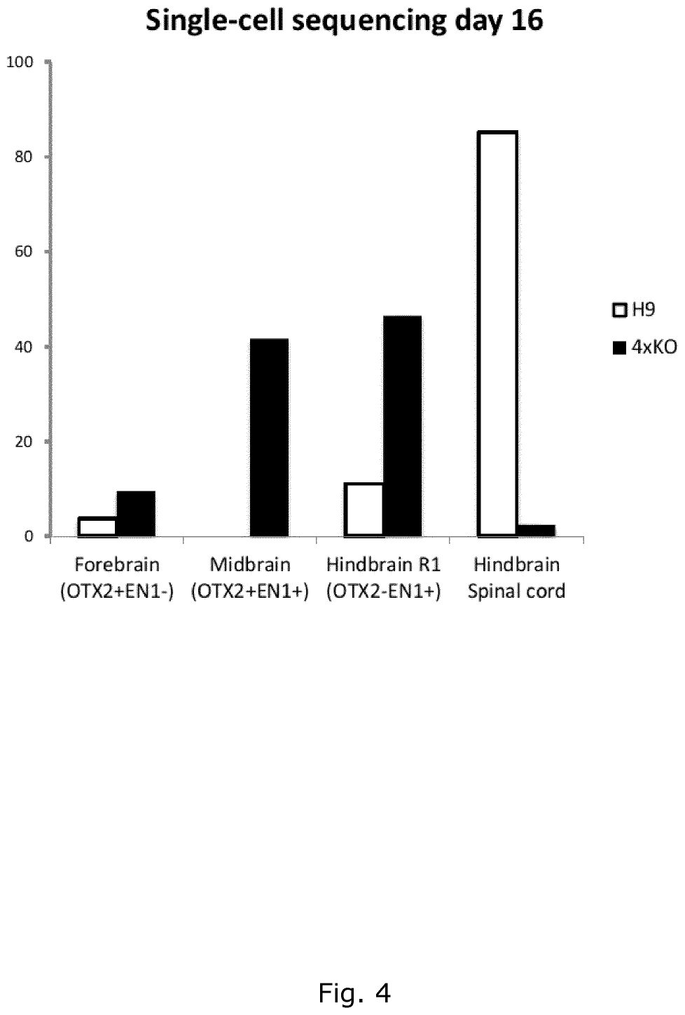

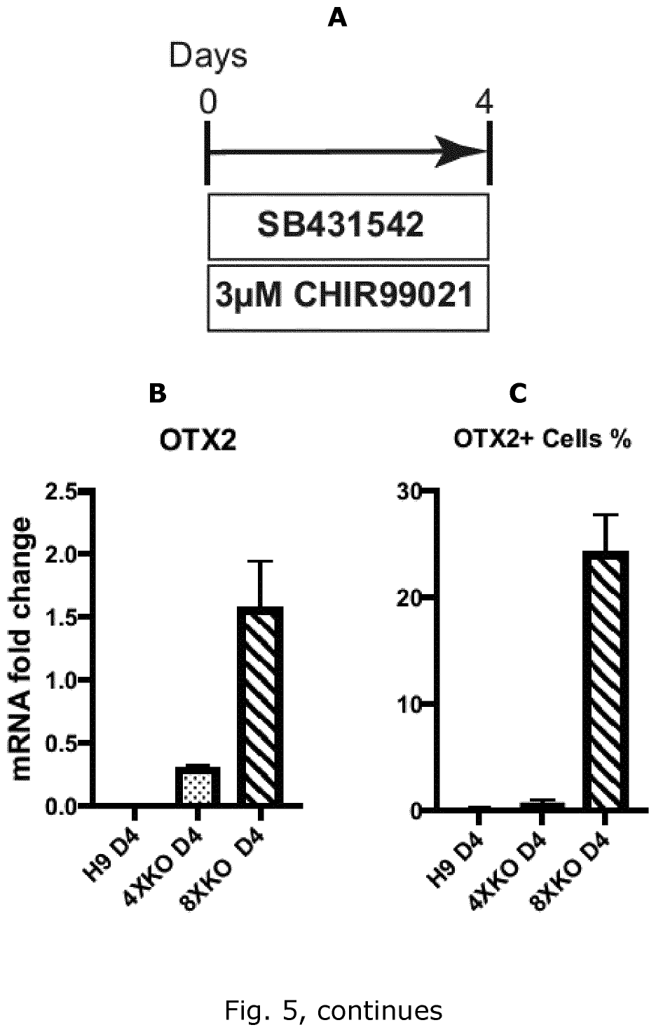

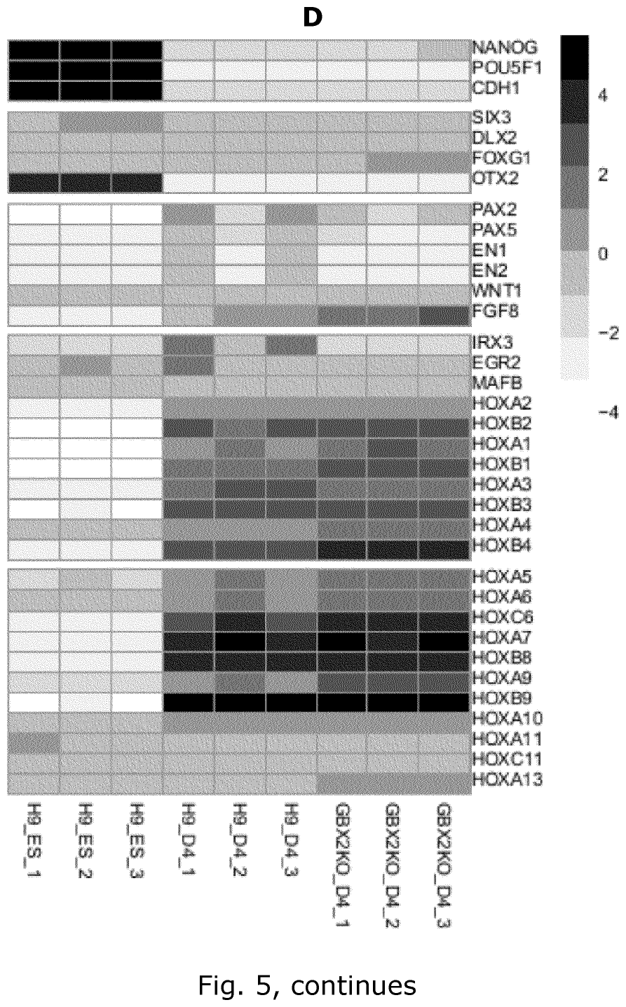

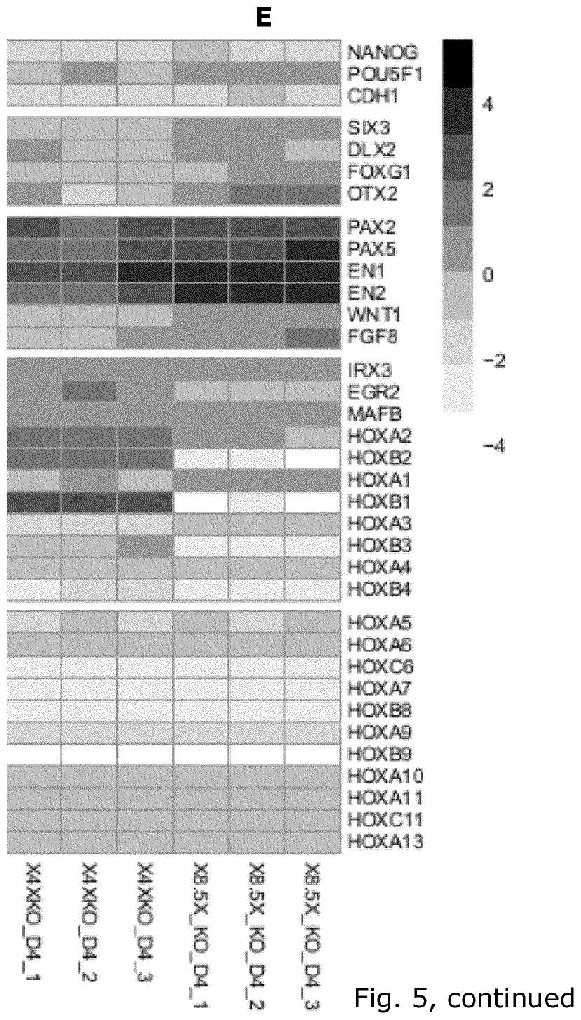

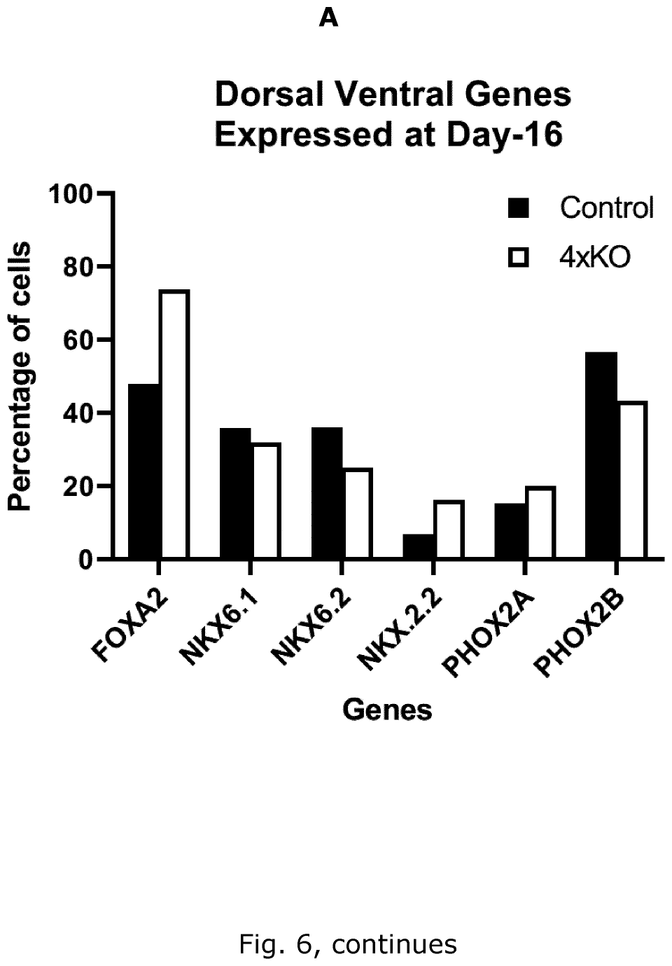

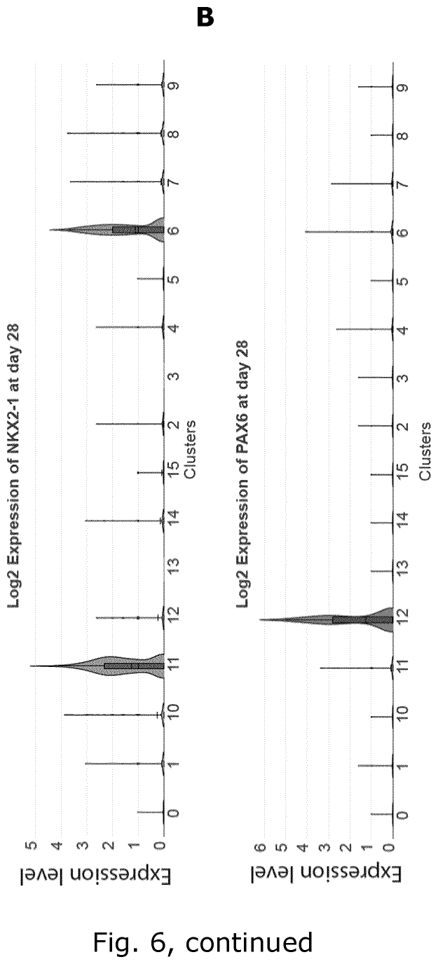

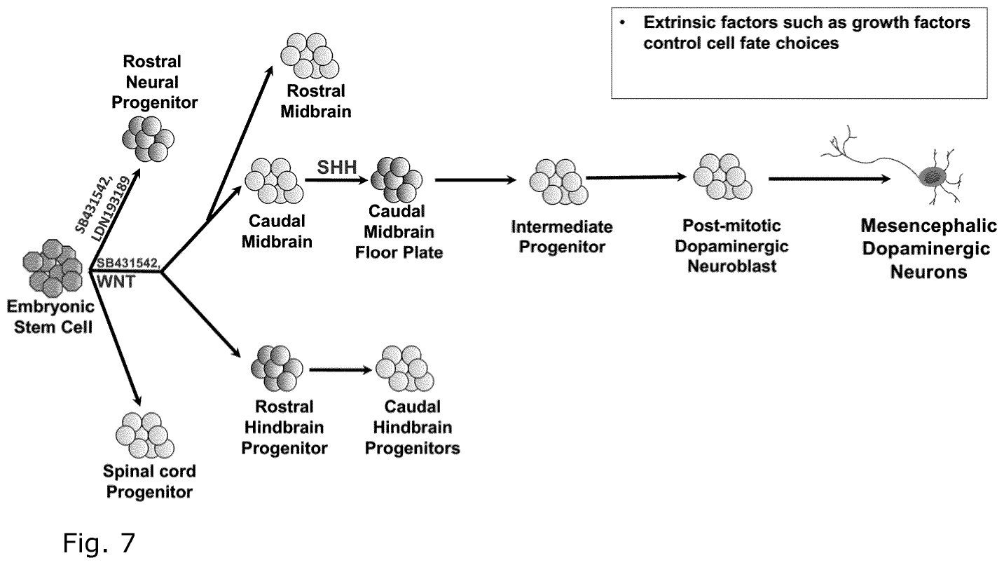

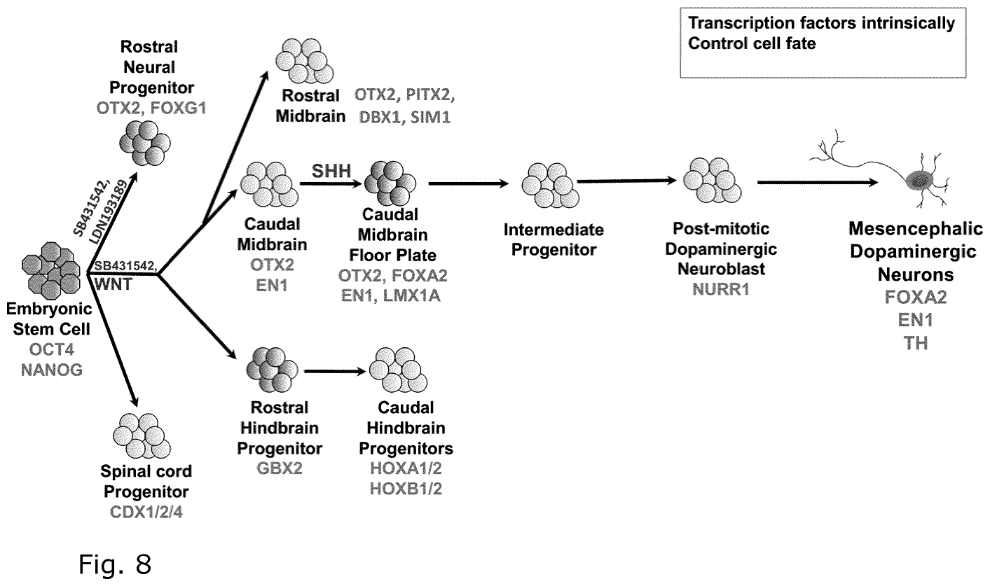

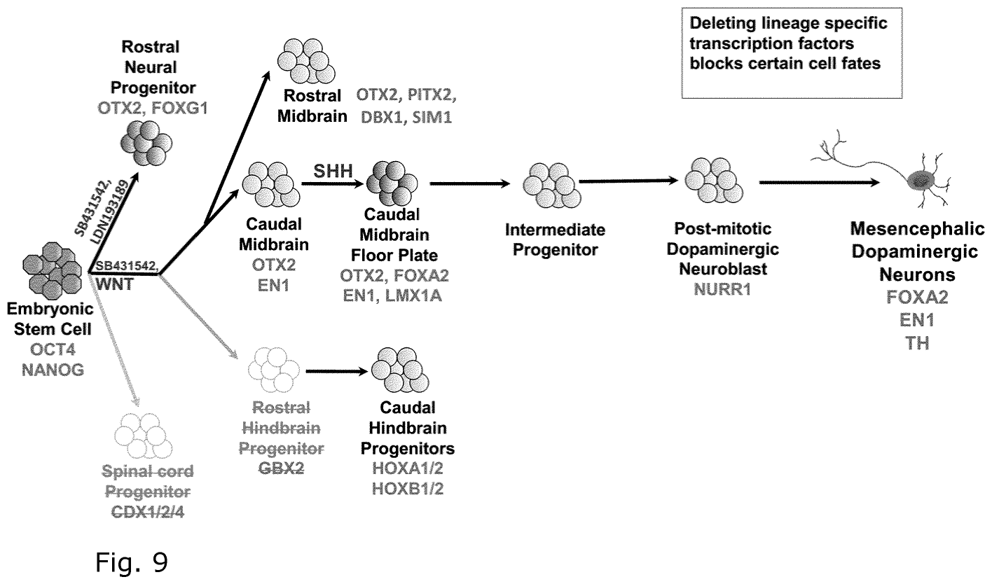

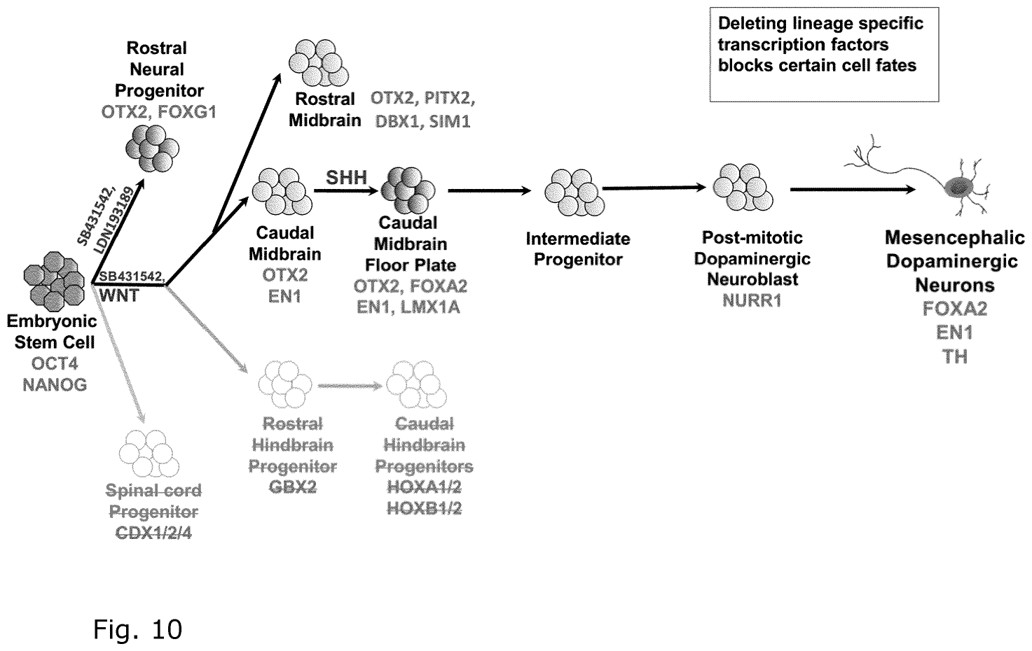

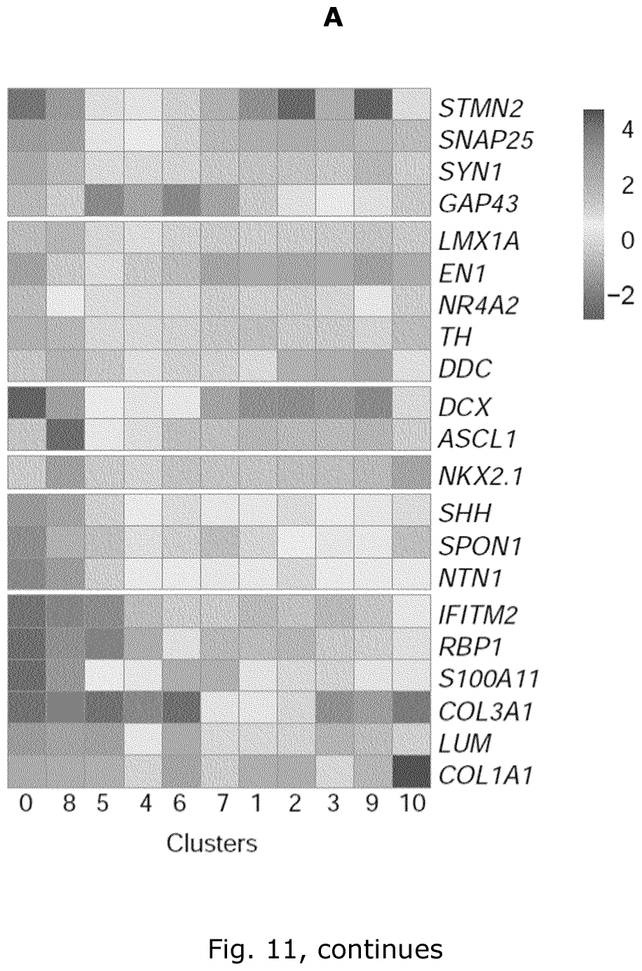

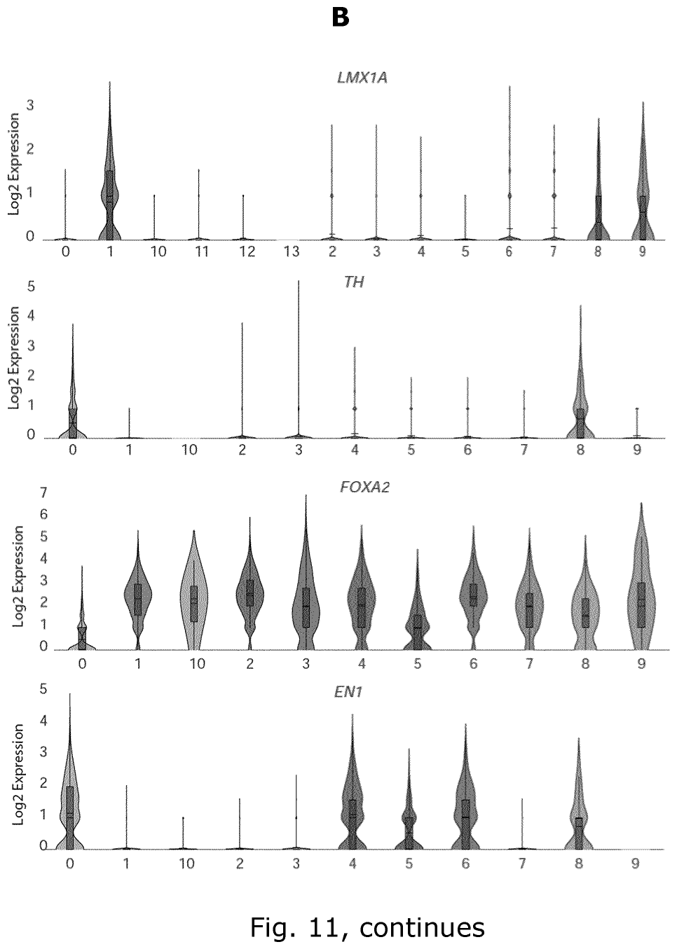

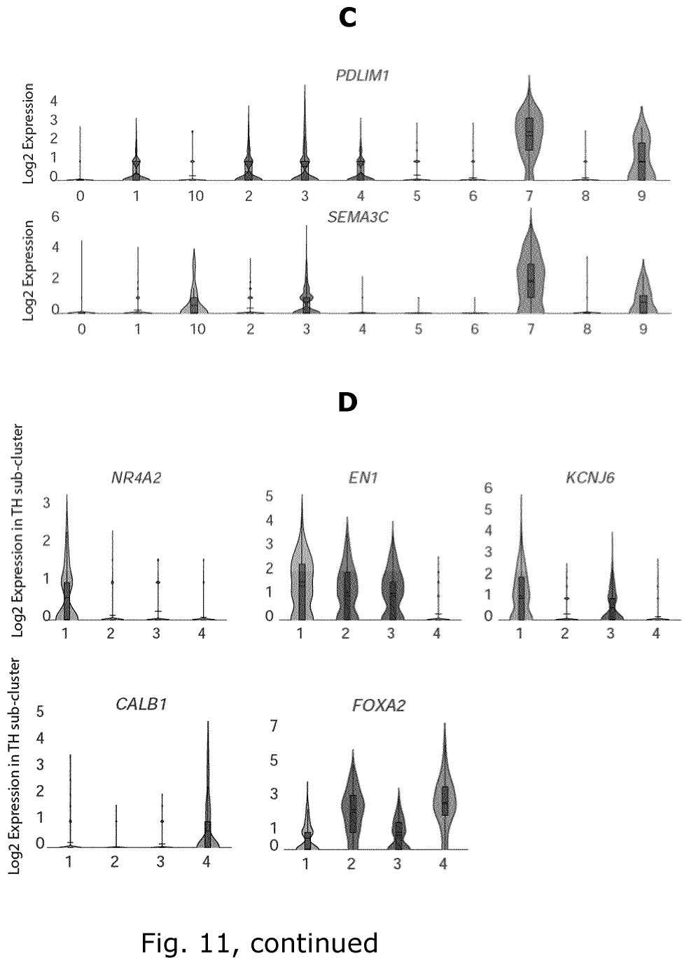

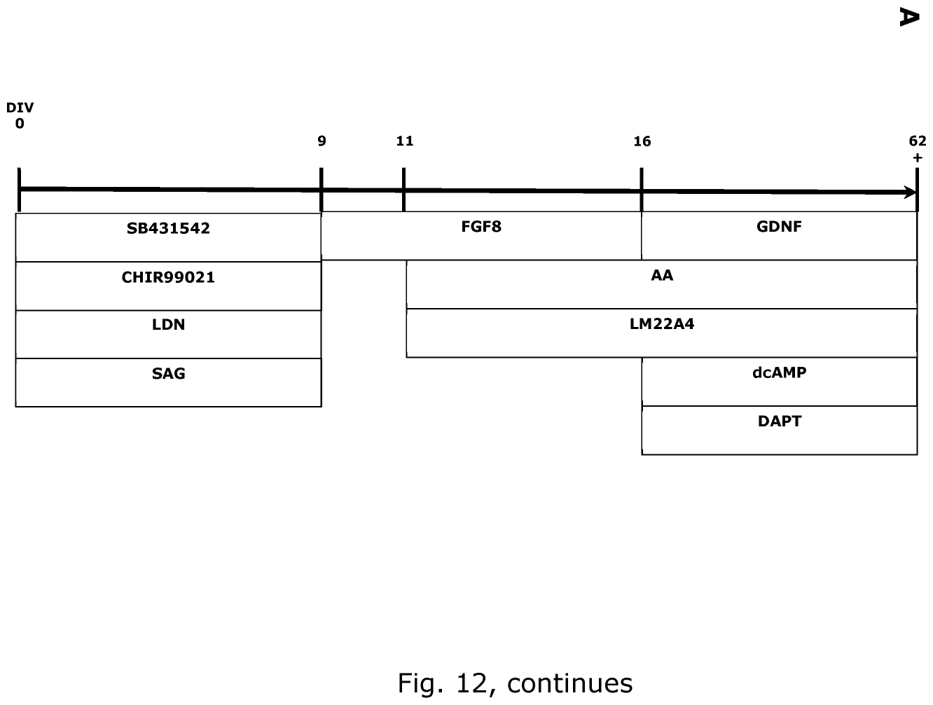

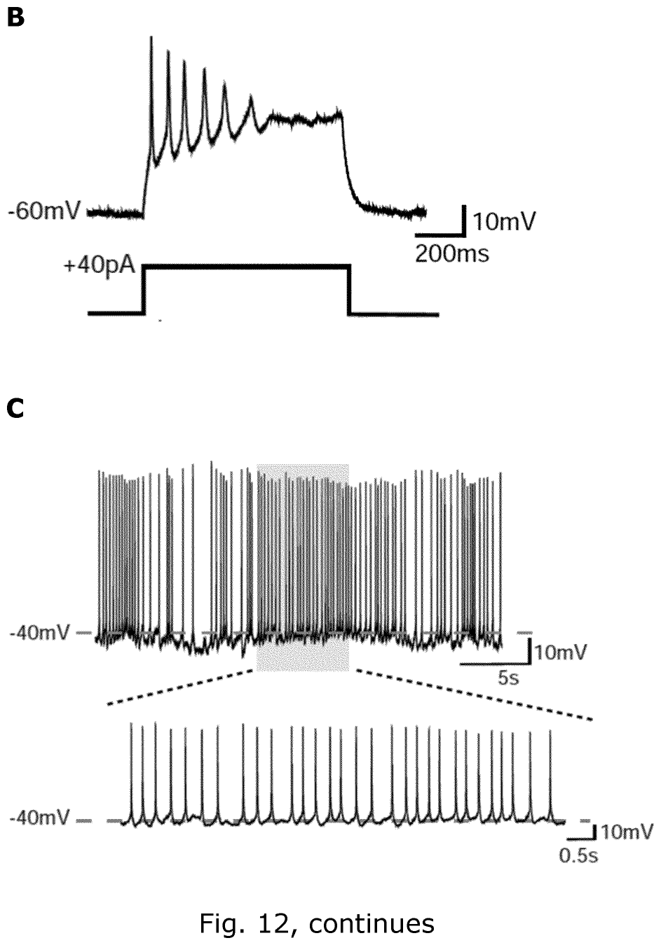

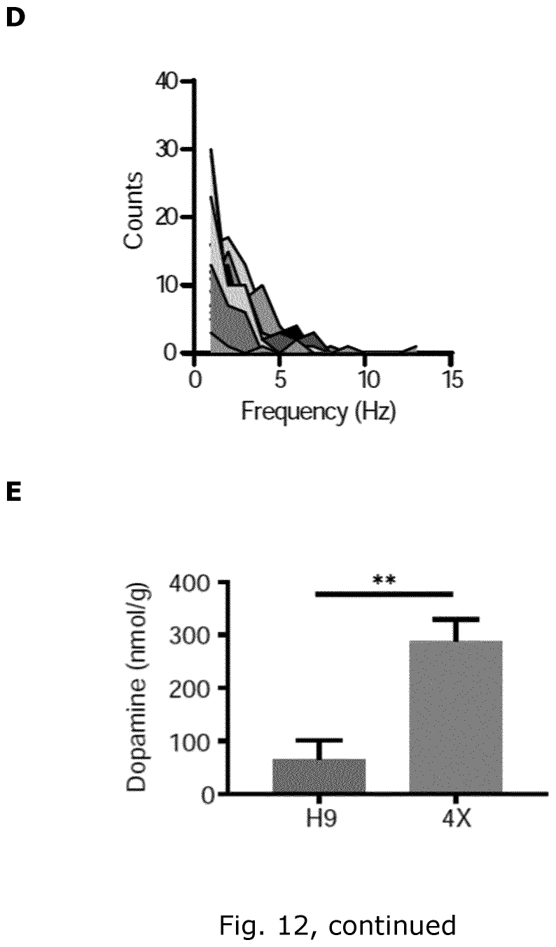

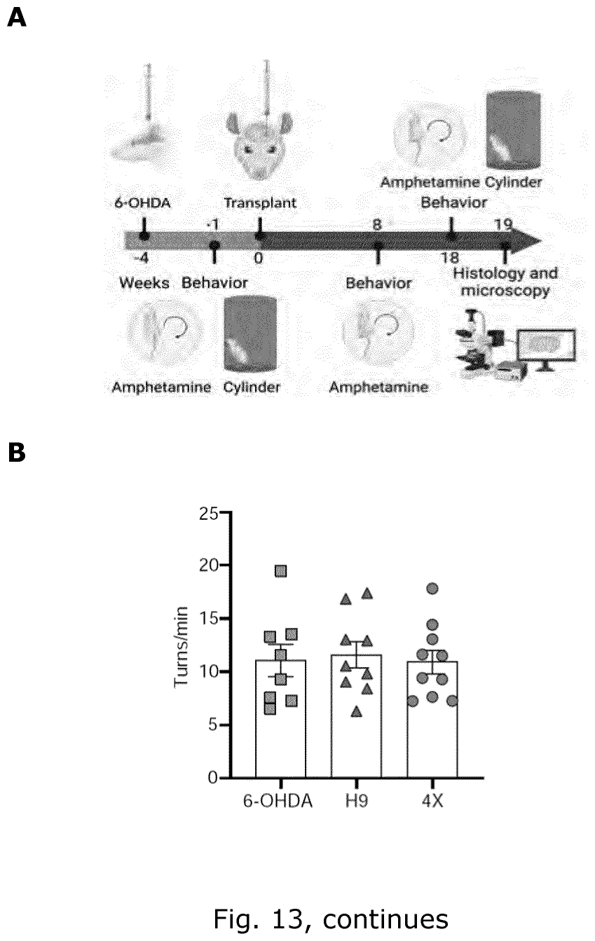

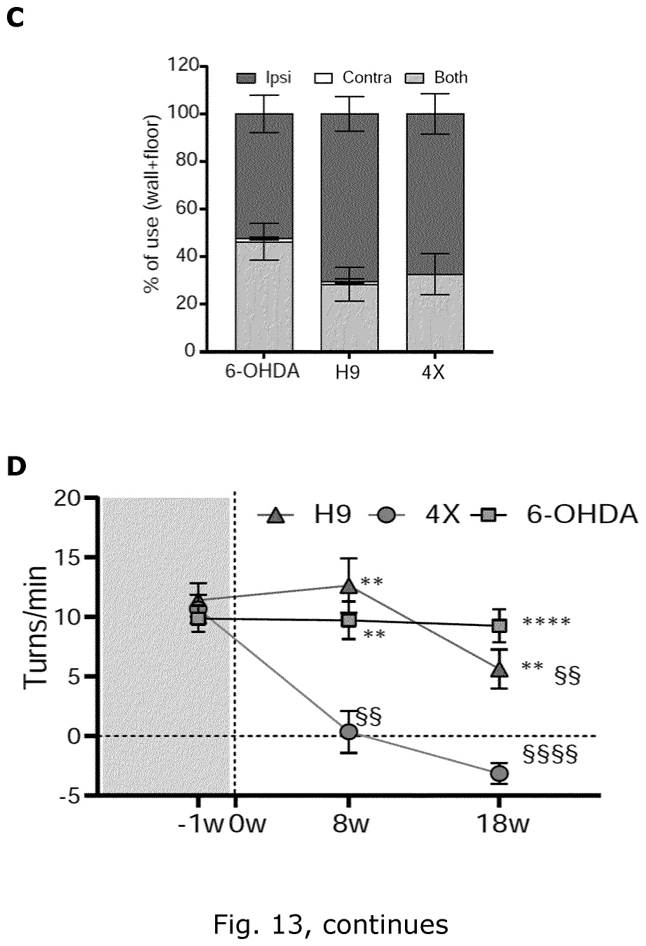

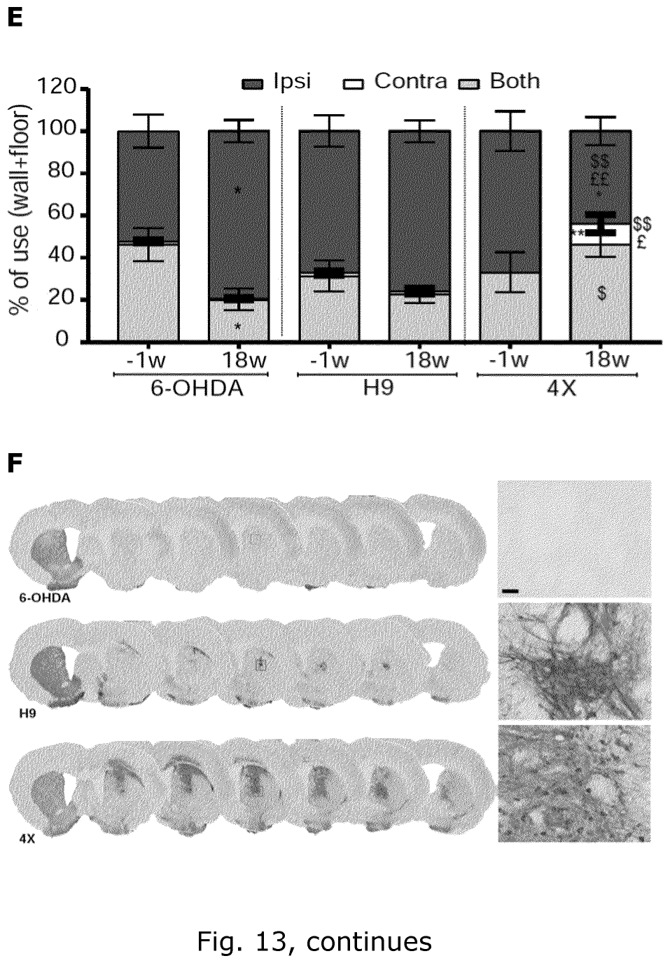

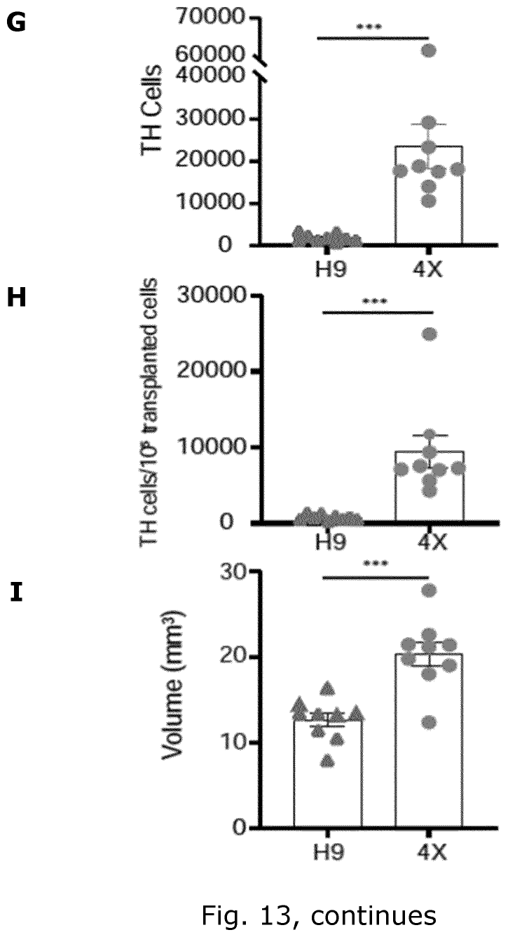

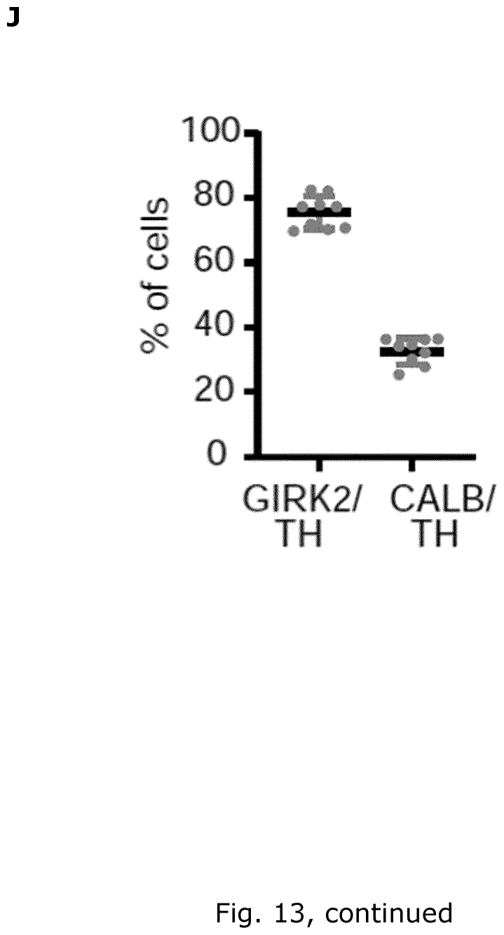

In another preferred embodiment, at least GBX2 and CDX2 are inactivated. By inactivating these genes, caudal neuroepithelial differentiation is prevented. Data for GBX2 is presented e.g. in FIGS. 1 and 2. Further, CDX2 is expressed is most of the spinal cord and is considered probably the most important out the three CDX genes.

In yet an embodiment, at least one of CDX1, CDX2 and CDX4 are inactivated, such as at least two of CDX1 CDX2 and CDX4 are inactivated, such as CDX1 and CDX2 are inactivated, such as CDX2 and CDX4 are inactivated or such as CDX1 and CDX4 are inactivated, or preferably such as at least CDX1, CDX2 and CDX4 are inactivated. By inactivating one or more of these genes caudal neural Progenitor cell differentiation is prevented.

In yet another embodiment, at least one of HOXA1, HOXA2, HOXB1 and HOXB2 are inactivated, such as at least two of HOXA1, HOXA2 and HOXB1 are inactivated, such as HOXA1 and HOXA2 are inactivated, such as HOXA2 and HOXB1 are inactivated or such as HOXA1 and HOXB1 are inactivated, or preferably such as at least HOXA1, HOXA2 and HOXB1 are inactivated. By inactivating one or more of these genes, hindbrain cell differentiation is prevented.

In yet another embodiment, the pluripotent stem cell according to any of the preceding claims, wherein at least GBX2, CDX1, CDX2, and CDX4 are inactivated. In examples 2-4, data for such a cell line is presented (4xKO cell line).

In another embodiment, at least GBX2, CDX1, CDX2 CDX4, GBX1, HOXA1, HOXA2 and HOXB1 are inactivated. In example 5, data for such a cell line is presented (8xKO cell line).

In yet an embodiment, at least the following genes are inactivated:
GBX2, CDX1, CDX2 and CDX4; or
GBX2, HOXA1, HOXA2 and HOXB1; or
GBX2, HOXA1, HOXA2, HOXB1 and HOXB2; or
GBX2, CDX2, HOXA1, HOXA2, HOXB1 and HOXB2.

In yet another embodiment, one or more of the following genes are inactivated:
PAX6, HOXB2, HOXA3, NKX6.1, NKX6.2, NKX2.1, NKX2.2, PAX6, BRN3A (POU4F1), PHOX2A, PHOX2B, PITX2, DBX1, and SIM1.

In example 6, the rationale behind the inactivation of these genes are further elaborated. In short, the listed genes represent cell types along the dorsal ventral axis.

The different genes can be inactivated by different methods. Thus, in an embodiment, the at least one gene is inactivated by gene knock-out or introduction of premature stop codons such as by CRISPR, or by gene silencing by preventing the transcription or translation of the gene, such as by siRNA, CRISPR inhibition or by introduction of a dominant-negative version of the gene, preferably using CRISPR. In the example section, inactivation has been performed by CRISPR, by introducing an indel mutation into the gene(s), which results in either a frameshift mutation or the deletion to part of the sequence resulting in the loss of one or more amino acids.

Different pluripotent stem cells may be used in the present invention. Thus, in an embodiment, the pluripotent stem cell is selected from the group consisting of an embryonic stem cell and an induced pluripotent stem cell, preferably of mammalian origin and even more preferably of human origin.

Pluripotent stem cells can be defined by expression patterns of different genes. Thus, in an embodiment, the pluripotent stem cell according to the invention is NANOG⁺, POU5F1⁺ (OCT4⁺) and/or SOX2⁺, preferably NANOG⁺, POU5F1⁺ (OCT4⁺) and SOX2⁺. The skilled person may identify other expressions patterns known in the art.

As also outlined above, it is noted that in an alternative aspect of the present invention the pluripotent stem cell is considered also to include astrocytes or reprogrammed/reprogrammable astrocytes. It is well known that astrocytes can be reprogrammed to differentiate in to other cell types such as neurons. For example, astrocytes have been demonstrated capable of being converted into mesencephalic dopaminergic neurons by the overexpression of specific genes (Rivetti et al., 2017). Also Corti et al. (Corti et al 2012) has shown dedifferentiation of human cortical astrocytes into the neural stem/progenitor phenotype to obtain progenitor and mature cells with a neural fate.

Thus, the restoration of multipotency from human astrocytes has potential in cellular reprogramming of endogenous central nervous system cells in neurological disorders. In combination with the present invention, this potential may be further explored.

Therefore, the method of enhancing the generation of dopaminergic neurons by knocking out genes to restrict their potential could also be applied to the conversion of (reprogrammed/reprogrammable) astrocytes to mesencephalic dopaminergic neurons.

It is to be understood that this aspect is also combinable with the other aspect of the invention.

### Use of the Pluripotent Stem Cells

The pluripotent stem cells according to the present invention can be used for different purposes. Thus, an aspect of the invention relates to the use of the pluripotent stem cell according to the present invention for cell lineage restricted cell differentiation of the pluripotent stem cell.

Another aspect relates to the use of the pluripotent stem cells according to the present invention for the generation of dopaminergic neuron progenitor cells or cell derivatives thereof.

In an embodiment, the dopaminergic neuron progenitor cells or cell derivatives thereof is selected from the group consisting of a dopaminergic neuron cell, such as a mesencephalic dopaminergic neuron cell, a mesencephalic dopaminergic progenitor neuron cell, a caudal midbrain progenitor cell, and a midbrain floor plate progenitor cell.

In another embodiment, said cell derivative thereof is a dopaminergic neuron cell, such as a mesencephalic dopaminergic neuron cell.

### Dopaminergic Neuron Progenitor Cells or Cell Derivatives Thereof

In particular, the present invention relates to dopaminergic neuron progenitor cells or cell derivatives thereof obtained from the lineage-restricted pluripotent stem cells according to the invention. Thus, yet an aspect relates to a dopaminergic neuron progenitor cell or cell derivatives thereof, differentiated from a pluripotent stem cell according to the invention. Again, as outlined in the example section, the lineage restricted stem cells of the invention can be efficiently be differentiated into dopaminergic neuron progenitor cells or cell derivatives thereof.

Thus, in yet another aspect, the invention relates to a dopaminergic neuron progenitor cell or cell derivatives thereof, characterized in that one or more of the genes selected from the group consisting of GBX2, CDX2, CDX1, CDX4, GBX1, HOXA1, HOXA2, HOXB1, HOXB2, HOXA3, NKX6.1, NKX6.2, NKX2.1, NKX2.2, PAX6, BRN3A (POU4F1), PHOX2A, PHOX2B, PITX2, DBX1, and SIM1 are inactivated.

In an embodiment, the dopaminergic neuron progenitor cell or cell derivatives thereof is characterized in that one or more of genes selected from the group consisting of GBX2, CDX2, CDX1, CDX4, GBX1, HOXA1, HOXA2 and HOXB1 are inactivated.

In another embodiment, the dopaminergic neuron progenitor cell or cell derivatives thereof is selected from the group consisting of a dopaminergic neuron cell, such as a mesencephalic dopaminergic neuron cell, a mesencephalic dopaminergic progenitor neuron cell, a midbrain floor plate progenitor cell, and a caudal midbrain progenitor cell.

In a related embodiment, said cell derivative thereof is a dopaminergic neuron cell, such as a mesencephalic dopaminergic neuron cell.

It is to be understood that the lists of inactivated genes shown for the aspect relating to pluripotent stem cells, also applies to this aspect.

The dopaminergic neuron progenitor cell or cell derivatives thereof can be defined by specific expression patterns. In an embodiment, the neuron (dopaminergic neuron progenitor cell) express FOXA2, LMX1A, EN1 and/or TH.

In a related embodiment, the dopaminergic neuron progenitor cell express (high levels of) FOXA2, LMX1A, OTX2, EN1, SPRY1, WNT1, CNPY1, PAX8, ETV5, PAX5, SP5, and/or TLE4.

It may be an advantage if the generated cells could be preserved before used. Thus, in an embodiment, neurons are cryopreserved.

### Medical Uses

The generated neuron cells according to the invention, may have different medical uses. Thus, an aspect of the invention relates to the dopaminergic neuron progenitor cell or cell derivatives thereof according to the invention, for use as a medicament, such as in stem cell therapy.

In another aspect, the invention relates to the dopaminergic neuron progenitor cell or cell derivatives thereof according to the invention, for use in the treatment, prevention or alleviation of neurodegenerative disorders.

In an embodiment, the dopaminergic neuron progenitor cell or cell derivatives thereof is grafted into the brain of the subject, preferably a mammalian subject, and more preferably a human subject.

In another embodiment, the neurodegenerative disorder is selected from the group consisting of Parkinson's disease, Lewy body disease, preferably Parkinson's disease. In a preferred embodiment, the neurodegenerative disorder is Parkinson's disease.

The dopaminergic neuron progenitor cell or cell derivatives thereof may be used for different forms of grafts. Thus, in an embodiment, the use is as an autograft, a xenograft or an allograft.

### In Vitro Use

The dopaminergic neuron progenitor cell or cell derivatives thereof according to the invention may also find use in vitro. Thus, an aspect of the invention relates to the use of the dopaminergic neuron progenitor cell or cell derivatives thereof according any to the invention in drug screenings assays. Such as drugs that prevent oxidative stress, prevent mitochondrial damage, promote the degradation of misfolded proteins, prevent the secretion of misfolded proteins and drugs that are neuroprotective.

### Method for Providing Lineage-Restricted Pluripotent Stem Cells

Yet an aspect of the invention relates to a method for providing a pluripotent stem cell according to the invention, the method comprising inactivating at least one gene selected from the group consisting of GBX2, CDX2, CDX1, CDX4, GBX1, HOXA1, HOXA2, HOXB1, HOXB2, HOXA3, NKX6.1, NKX6.2, NKX2.1, NKX2.2, PAX6, BRN3A (POU4F1), PHOX2A, PHOX2B, PITX2, DBX1, and SIM1 in a pluripotent stem cell.

In an embodiment, one or more of genes selected from the group consisting of GBX2, CDX2, CDX1, CDX4, GBX1, HOXA1, HOXA2 and HOXB1 are inactivated in a pluripotent stem cell.

Again, it is to be understood that the lists of inactivated genes shown for the aspect relating to pluripotent stem cells, also applies to this aspect.

In yet an embodiment, the provided pluripotent stem cell(s) comprising at least one inactivated gene, is limited in its capability to differentiate into cell lineages different from dopaminergic neuron progenitor cells or cell derivatives thereof.

In a related embodiment, the provided pluripotent stem cell comprising at least one inactivated gene, is limited in its capability to differentiate into cell lineages selected from the group consisting of caudal neural progenitor cells, spinal cord progenitor cells, hindbrain progenitor cells, metencephalon progenitor cells, and myelencephalon progenitor cells.

In an embodiment, the at least one gene is inactivated by gene knock-out or introduction of premature stop codons, such as by CRISPR, or by disrupting the transcription or translation of the gene, such as by gene silencing, by siRNA or by CRISPR inhibition or by expression of a dominant-negative version of the gene, preferably by CRISPR.

A further aspect of the invention relates to a pluripotent stem cell obtained/obtainable by a method according to the invention.

### Method for Providing Dopaminergic Neuron Progenitor Cells

Yet a further aspect of the invention relates to a method for providing dopaminergic neuron progenitor cells or cell derivatives thereof the method comprising
providing a pluripotent stem cell according to the invention; and
differentiating the provided pluripotent stem cell into dopaminergic neuron progenitor cells or cell derivatives thereof.

An advantage of the method according to the present invention, is that the amounts of different components in the cell differentiation media may be less restricted. For example, as shown in example 3, the concentration of CHIR99021 is less restricted. Thus, the desired cell type can still be generated even when sub-optimal concentrations of growth factors or small molecules are used.

In yet an embodiment, the provided dopaminergic neuron progenitor cells or cell derivatives thereof are cryopreserved.

In another embodiment, the dopaminergic neuron progenitor cells or cell derivatives thereof are selected from the group consisting of a dopaminergic neuron cell, such as a mesencephalic dopaminergic neuron cell, a mesencephalic dopaminergic progenitor neuron cell, a midbrain floor plate progenitor cell, and a caudal midbrain progenitor cell.

In a further embodiment, said cell derivative thereof is a dopaminergic neuron cell, such as a mesencephalic dopaminergic neuron cell.

In yet another embodiment, the method is performed in vitro and/or ex vivo.

### Other Aspects of the Invention

A further aspect of the invention relates to a method for treating or alleviating a subject suffering from a neurodegenerative disorder, the method comprising grafting the dopaminergic neuron progenitor cells or cell derivatives thereof according to the invention, into the brain of the subject.

It should be noted that embodiments and features described in the context of one of the aspects of the present invention also apply to the other aspects of the invention.

All patent and non-patent references cited in the present application, are hereby incorporated by reference in their entirety.

The invention will now be described in further details in the following non-limiting examples.

### EXAMPLES

### Example 1-Materials and Methods

### Culture of Human Pluripotent Stem Cells

Human embryonic stem cells (hESCs; H9 cell line, WiCell) were maintained on irradiated human fibroblasts in KSR media consisting of DMEM/nutrient mixture F-12, supplemented with non-essential amino acids (NEAA) 1%, glutamine 2 mM, β-mercaptoethanol 0.1 mM, 0.5% pen/strep and 20% knockout serum replacement. KSR media was supplemented with FGF2 (15 ng/ml; Peprotech) and Activin A (15 ng/ml; R&D Systems). Every seven days cells were manually passaged and fragments transferred to a freshly prepared gelatin-coated dish containing irradiated fibroblasts.

### Differentiation to Day 4 and Day 11 Caudal Neural Progenitor

hESCs were differentiated to caudal neural progenitors as described previously (Denham et al., 2015). Briefly, hESC fragments were cut from colonies growing on feeders (CCD-1079Sk, ATCC) and plated onto vitronectin-coated plates in N2B27 media containing 1:1 of Neurobasal medium (NBM) and DMEM/F-12 supplemented with N2 supplement 1%, B27 Supplement Minus Vitamin A 1%, insulin/transferrin/selenium-A (ITS-A) 1%, glucose 0.3%, glutamax supplement 1%, penicillin/strepto-mycin 0.5% (all from Life Technologies). Media was supplemented with SB431542 (SB; 10 UM, Tocris Bioscience) and LDN193189 (LDN; 100 nM, Stemgent) and CHIR99021 (CHIR; 3 μM, Stemgent) for 4 days. For day 11 caudal neural progenitor, cell were cultured as above until day 4 and then colonies were dissected into 0.5 mm pieces and cultured in suspension in low-attachment 96-well plates (Corning, Corning, NY, USA, www.corning.com) in N2B27 medium supplemented with FGF2 (20 ng/ml; Peprotech) and 400 nM SAG (Millipore).

### Differentiation to Mesencephalic Dopaminergic Neurons

Generation of mesDA neurons was achieved by implementing previously described protocols, with minor modifications (Nolbrant et al., 2017). Briefly, from day 0 to day 9 cells were grown in N2B27 media supplemented with 10 UM SB431542, CHIR99021 (0.5 to 1 μM), 0.1 μM LDN-193189 (Stemgent), and 400 nM SAG (Millipore). At day 4, colonies were cut into fragments and cultured in suspension. At day 9 to day 11, the supplements in the media was changed to FGF8 (100 ng/ml; R&D Systems). From day 11 media was supplemented with FGF8 (100 ng/ml), LM22A4 (2 μM), and Ascorbic Acid (200 μM; Sigma). From day 16 onwards cell were dissociated with accutase and grown on culture plates coated with polyornithine, fibronectin, and laminin (all from Sigma). Neural differentiation media consisting of B27 1%, Pen/Strep 25 U/mL, Glutamax 0.5%. NDM was supplemented with 200 UM Ascorbic acid, LM22A4 (2 M), 1 μM DAPT (Tocris bioscience), GDNF (10 ng/ml), dcAMP (500 UM). The media was changed every second day until the end of the experiment. Alternatively, at day 16 culture were maintained in suspension to generate organoids.

### Lentivirus Production

Third generation lentiviruses were generated as described previously (Gill and Denham, 2020). In brief, five lentiviral plasmids, pLV-4gRNA-GBX2-RFP, pLV-hUbC-GBX2-CDX124-Cas9-T2A-GFP, PLV-HOXA1+2-HOXB1-hUbC-dsRED, pLV-Puro-U6-GBX1-G3-U6-GBX1-G1 and lentiCas9-Blast (Addgene #52962), were used to produce lentiviruses.

### Generation of Knockout Cell Lines

To generate the gene knockout cell lines, the multiplex CRISPR/Cas9-based single lentiviral vector system was used (Kabadi et al., 2014). The construction of the lentiviral plasmids containing the guide sequences required the use of the Golden Gate cloning method. Specifically, the following donor plasmids were used ph7SK-gRNA, phU6-gRNA, pmU6-gRNA, phH1-gRNA and the destination vectors pLV hUbC-Cas9-T2A-GFP and pLV GG hUbC-dsRED (Addgene; 53189, 53188, 53187, 53186, 53190, 84034). The donor plasmids were digested with BbsI and a pair of single-stranded oligos containing the genomic target sequences for the genes were annealed together and cloned into the plasmids. A set of four Golden Gate compatible donor vectors were then selected and used to generate the final destination vector. Briefly, the selected donor vectors and the destination vector were all digested with BsmBI and fragments ligated together with T4 ligase, transformed and colonies selected using ampicillin. The final destination vectors generated were pLV-GBX2-CDX1+2+4-hUbC-Cas9-T2A-GFP and pLV-HOXA1+2-HOXB1-hUbC-dsRED. For GBX1, the lentiviral dual guide puromycin vector pLV-Puro-U6-GBX1-G3-U6-GBX1-G1 was designed and purchased from Vectorbuilder (vector ID: VB190322-1078gcv), and guide sequence of Table 1.

In brief, the GBX2 knockout cell line, H9 cells were transduced with LV-4gRNA-GBX2-RFP and lentiCas9-Blast, and after three days, transduced cells were selected using 10 μg/ml blasticidin for 6 days. FACS was then used to separate single RFP-positive cells in a 96-well plate using the 561 nm laser on a FACSAriaIII (BD Biosciences, San Jose, CA). Indels at the corresponding target sites in the clones were analyzed by genomic PCR. To generate the 4X knockout cell line, H9 cells were infected with LV-hUbC-GBX2-CDX124-Cas9-T2A-GFP, and after 7 days, single GFP-positive cells were sorted by FACS. The 8× knockout cell line was generated in a similar manner. Allele-specific mutations were confirmed using whole-exome sequencing. Whole-exome sequencing and mapping were performed by BGI (BGI, Copenhagen). Integrated Genome Browser V 2.10.0 was used to identify allele-specific mutations. To identify large deletions that could not be mapped by the alignment tools, individual sequencing reads were extracted from the FastQ files using Grep and manually analyzed.

Based on the above, the skilled person could design knockout cell lines of the other relevant genes listed but not tested here.

TABLE 1
Summary of the Guide RNA target sequences used
to generate knockout cell.

| Cell | | Genomic target sequence + | SEQ |
|---|---|---|---|
| line | Gene | PAM of Guide RNA | ID NO: |
| 4xKO | GBX2 | AAAGGTGGAAGACGACCCGA AGG | 1 |
| | CDX1 | CTACACCGACCACCAACGCC TGG | 2 |
| | CDX2 | GTACACGGACCACCAGCGGC TGG | 3 |
| | CDX4 | CCTGGGCCTTTCCGAGAGAC AGG | 4 |
| 8xKO | GBX2 | AAAGGTGGAAGACGACCCGA AGG | 1 |
| | CDX1 | CTACACCGACCACCAACGCC TGG | 2 |
| | CDX2 | GTACACGGACCACCAGCGGC TGG | 3 |
| | CDX4 | CCTGGGCCTTTCCGAGAGAC AGG | 4 |
| | GBX1 | TAAATGCTGTGCGGCGCCGT CGG | 5 |
| | HOXA1 | GTACCTGACGCGCGCCCGCA GGG | 6 |
| | HOXA2 | GAATCCCTGGAAATCGCCGA TGG | 7 |
| | HOXB1 | GGTGGAGATTGCCGCCACCC TGG | 8 |

### QPCR and NanoString

QPCR and NanoString experiments were performed as commonly known to the persons skilled in the art. In brief, RNA was extracted using the Qiagen RNeasy mini kit and treated with DNase I according to a standard protocol. cDNA was generated from 500 ng of total RNA using Superscript III and random primers following the manufacturer's instructions. For QPCR, TaqMan Universal Master mix II without UNG and TaqMan probes were used. NanoString experiments were performed using the NanoString nCounter SPRINT (NanoString Technologies) according to the manufacturer's instructions. Briefly, 200 ng of total RNA was used. Reporter probes were hybridized for 20 hours at 65° C. A custom designed NanoString CodeSet consisting of a panel of capture and reporter probes designed to target 100 nucleotides of the gene of interest and a panel of housekeeping genes was used. RNA expression data were normalized to the expression of housekeeping genes.

### Immunofluorescence

Immunofluorescence were performed as commonly known to the persons skilled in the art. In brief, the cells cultured on glass coverslips or suspended in culture plates as spheroids were collected. The samples were washed with PBS two times, fixed in 4% paraformaldehyde (PFA) in PBS at 4° C. for 15 min (glass coverslips) or 2 hours (spheroids), and washed 3 times with PBS for 10 min each. The spheroids were transferred to 20% sucrose in PBS, incubated at 4° C. overnight and embedded in OCT (Tissue-Tek). Sections were cut at a thickness of 10 μm using a cryostat (Crostar NX70) at −20° C. The coverslips and sections were incubated in 0.25% Triton X in PBS (PBST) for 10 min and blocked in 5% donkey serum (Almeco) in PBST for 1 hour at room temperature. The following primary antibodies were applied overnight at 4° C.: goat anti-OTX2 (1:500, R&D Systems, cat #AF1979), mouse anti-CDX2 (1:200, BioGenex, cat #MU392-UC), mouse anti-Engrailed1 (EN1, 1:40, DSHB, cat #4G11-s), rabbit anti-EN1 (1:50, Merck, cat #HPA073141), rabbit anti-FOXA2 (1:500, Cell Signaling, cat #8186), goat anti-FOXA2 (1:200, R&D Systems, cat #AF2400), rabbit anti-LMX1A (1:5000, Millipore, cat #AB10533), mouse anti-TH (1:2000, Millipore, cat #MAB318), rabbit anti-TH (1:1000, Pel Freez, cat #P40101-150), rabbit anti-GIRK2 (1:500, Alomone, cat #APC-006), mouse anti-CALB1 (1:5000, SWANT, cat #300), rabbit anti-Collagen3A1 (1:1000 NovusBio, cat #NB120-6580), sheep anti-hCOL1A1 (1:200, R&D Systems, cat #AF6220), and mouse anti-HNA (1:200, Abcam, cat #ab191181). After the cells were washed with PBST three times for 10 min each, corresponding secondary antibodies (1:200, Jackson ImmunoResearch Laboratories or 1:1000, Invitrogen) were applied for one hour at room temperature. After the secondary antibodies were removed, the cells were washed three times with PBST for 10 min each in the dark. The nuclei were counterstained with DAPI (1 μg/ml, Sigma) and rinsed with PBS three times for 5 min each. The slides or coverslips were mounted with PVA-DABCO. Images were captured with a confocal microscope (Zeiss LSM 780) and Zen software.

### Flow Cytometry Analysis

Flow cytometry analysis were performed as commonly known to the persons skilled in the art. In brief, the cells were washed two times with PBS—and dissociated with Accutase to obtain single cells. The cells were centrifuged at 300×g for 4 min and resuspended in 4% PFA for 10 min at room temperature. Then, the cells were washed with PBS−, centrifuged, resuspended in PBST, centrifuged again, and blocked in 5% donkey serum for 30 min at room temperature. Primary antibodies in blocking solution were added to the cells, and the cells were incubated for 2 hours at room temperature. The cells were washed once with PBST, resuspended in secondary antibodies in blocking solution and incubated for 30 min at room temperature in the dark. The cells were washed with PBST overnight at 4° C. and resuspended in PBS for flow cytometry using a NovoCyte Quanteon analyzer (Acea Biosciences Inc., Santa Clara, CA). The data were analyzed with FlowJo software (v. 10, Ashland, OR).

### Quantification of Immunofluorescence Images

Quantification of immunofluorescence images were performed as commonly known to the persons skilled in the art. In brief, the percentages of OTX2/DAPI double-positive, GIRK2/TH double-positive and CALB1/TH double-positive cells, either in culture or within a graft, were quantified with ImageJ software (1.53) by semiautomatic object-based colocalization analysis (Lunde et al., 2020). The Colocalization Image Creator Plugin was used to process the multichannel immunofluorescence images into multichannel binary and grayscale output images. Binary output images were generated by processing input channels for ImageJ filters that applied an automatic local intensity threshold, radius outlier removal, watershed segmentation, eroding, hole filling, Gaussian blurring and maximum algorithms. Binary objects of an inappropriately small size were further removed from the output images via a defined minimum area size. To improve the visualization of the colocalization signals, the object overlap was restricted to the nuclei of the cells. The accuracy of the binary object segmentation was visually verified via grayscale output images. Once verified, the binary objects, representing either individually labeled or colabeled cells, were quantified automatically using the Colocalization Object Counter plugin. All immunofluorescence images were analyzed with conserved binary object segmentation settings. A minimum of 4 random fields captured at 20× and 63× were used for the quantification of OTX2 positive cells in culture. Quantification of GIRK2/TH double-positive and CALB1/TH double-positive cells within the graft was performed blindly by analyzing 4 nonoverlapping images taken at 20×from 2 sections per graft per animal.

### RNA Sequencing and Data Analysis

RNA sequencing and data analysis were performed as commonly known to the persons skilled in the art. In brief, library construction, sequencing and initial data filtering, including adaptor removal, were performed by the BGI Europe Genome Center. Total RNA was subjected to oligo dT-based mRNA enrichment. Sequencing of 100 bp paired-end reads was performed on the DNBseq platform. More than 20 million clean reads were obtained per sample. The reads were aligned to the Human genome build hg38 (Ensemble release 92) using HISAT2 aligner (v2.1.0). Transcript quantification was performed using FeatureCount (v1.6.4), and the read counts were normalized for effective gene length and sequencing depth to yield transcripts per kilobase million (TPM). Differentially expressed genes were identified from count tables using edgeR (v3.32). Centering and univ variance scaling were applied to TPM values to construct heatmaps and perform principal component analysis (PCA) by Clustvis using SVD with imputation (Metsalu et al., 2015).

### Single-Cell RNA-Seq and Data Analysis

Single-cell RNA-seq and data analysis were performed as commonly known to the persons skilled in the art. In brief, on days 16, 28 and 62, cultured cells were dissociated into single cells using Accutase. On day 16, neurospheres (n=10 biological replicates per cell line) were pooled together, and on day 28, four biological replicates per cell line were pooled together, and on day 62 four biological replicates per cell line were pooled together. To construct the library, the 10× Genomics Chromium Next GEM Single Cell 3′ kit v 3.1 was used according to a standard protocol. Each of the 6 groups (day 16 H9 cells, day 16 4X cells, day 28 H9 cells, day 28 4X cells, day 62 H9 cells and day 62 4X cells) was run in separate lanes of the Chromium controller, and a total of 8,000 cells were loaded per lane. Next-generation sequencing was performed on an Illumina NovaSeq instrument. The Cell Ranger Single-Cell Software Suite (v 3.1.0) was used for sample demultiplexing, barcode processing and single-cell 3′ gene counting. The reads were aligned to the human GRCh38 reference genome. Further analysis, including quality filtering, dimensionality reduction, and application of standard unsupervised clustering algorithms, was performed using the Seurat R package (v 3.2.1). To exclude outlier cells, the number of genes expressed in each cell was plotted for each sample to select the optimal allowed minimum number of genes per cell. The minimum numbers of genes per cell were set to 3000 for day 16_H9 cells, 2000 for day 16_4X cells, 3000 for day 62_H9 cells, and 3000 for day 62_4X cells. Cells with a high percentage of reads mapped to mitochondrial genes were also removed. For day 16 and 28 samples, all cells with more than 10% mitochondrial RNA were removed; for day 62, the limit was 15%. The R package DoubletFinder (v.2.0.3) was used to remove cell doublets from the single-cell transcriptome data, with the expected percentage of doublet cells being set at 7.5%. The single-cell data were normalized by dividing the gene counts of each cell by the total counts for that cell, multiplying by a scaling factor of 10,000, and natural-log transforming the result. Dimensionality reduction was performed using the UMAP technique. Clustering was performed by Seurat's graph-based clustering approach using the FindClusters function, with the resolution set to 0.6. Various single-cell plots were generated using Seurat in R.

### Electrophysiology

Electrophysiological recordings of 4X cells were performed at 80-84 DIV. 4X cells cocultured with astrocytes on 13 mm Ø coverslips were transferred to the recording chamber following progressive transition from culture medium to artificial cerebrospinal fluid (aCSF) by adding five drops (200 μL each) of aCSF to the cultured medium over 20 s. After being transferred into the recording chamber, the coverslips were continuously perfused at room temperature with aCSF containing (in mM) 119 NaCl, 2.5 KCl, 26 NaHCO₃, 1 NaH2PO₄, 11 D-glucose, 2 CaCl₂), and 2 MgCl₂ (adjusted to pH 7.4).

The recording chamber was mounted on an upright microscope (Scientifica) linked to a digital camera (QImaging Exi Aqua). The 4X cells were visualized using a 63× water-immersion objective (Olympus, LumiPlan). The cells selected for electrophysiological recordings exhibited a neuron-like morphology with fine branching neurites. Clusters of amassed cells were avoided. Acquisitions were performed in whole-cell configuration in current-clamp mode using Clampex 10.6 software connected to a Multiclamp 700B amplifier via a Digidata 1550A digitizer (Molecular Devices). The data were low-pass filtered at 200 Hz and digitized at 10 kHz, and the whole-cell capacitance was compensated. Patch pipettes (resistance of 5-10 MOhm) were filled with an internal solution containing (in mM) 153 K-gluconate, 10 HEPES, 4.5 NaCl, 9 KCl, 0.6 EGTA, 2 MgATP, and 0.3 NaGTP. The pH and osmolarity of the internal solution were close to physiological conditions (pH 7.4, osmolarity of 297 mOsm). The access resistance of the cells in our sample was ˜ 30 MOhm. Among the recordings of 30 neurons that were obtained, 16 were kept for analysis. The rest of the recordings were from neurons that either were nonrespondent to depolarizing steps (putative astrocytes), were unstable, or did not exhibit spontaneous activity; therefore, these recordings were discarded from the analysis.

Spontaneous excitatory postsynaptic potentials (sEPSPs) were recorded in current-clamp gap-free mode (clamped at −45 mV). Current-clamp recordings (at −60 mV) of evoked action potentials were performed by applying a repetitive current pulse (800 ms) with an incremental amplitude (20 pA).

Data analysis was performed using Clampfit 10.6 software (Molecular Devices). To visualize the distribution of the firing frequency, the number of spontaneous spikes per second in current-clamp mode was counted over a one-minute period using the threshold tool of Clampfit software and classified in bins with a width equal to 1 (corresponding to 1 Hz). The data were visualized using the frequency distribution mode of GraphPad Prism V9 software.

### HPLC Analysis of Dopamine Content

At 80 DIV, 1-2 organoids per sample were collected and homogenized in 100 μl of 0.2 M HClO₄. Then, the samples were centrifuged, and the supernatant was collected and spun through a 0.2 μm spin filter (Costar Spin-X, Merck) at 14000× g at 4° C. for 1 min and loaded into an HPLC system (Thermo Scientific Ultimate 3000). The mobile phase was 12.5% acetonitrile buffer (pH 3.0, 86 mM sodium dihydrogen phosphate, 0.01% triethylamine, 2.08 mM 1-octanesulfonic acid sodium salt, and 0.02 mM EDTA). The flow rate of the mobile phase was adjusted to 1.5 ml/min. The dopamine level was calculated using a standard curve generated using external DA standards (the standard curve coefficient of determination was 0.99946). Dopamine content was then normalized to the protein concentration and is expressed in nmol/g.

### Preparation of Cells for In Vivo Transplantation

For each batch of single cells, ten neurospheres at 16 DIV from each cell line (H9 and 4X) were collected and washed twice with PBS. Then, 500 μl of Accutase (supplemented with 100 μg/ml DNase) was added, and the cells were incubated for 10 min at 37° C. The neurospheres were first pipetted with a 1 ml pipette followed by a 200 μl pipette to yield a single-cell solution. Five hundred microliters of washing medium (DMEM/F12 supplemented with 1% human serum albumin) was added, and the cells were spun down at 400×g for 5 min at room temperature. The cell pellets were resuspended at a concentration of 100,000 cells/μl in HBSS (supplemented with 100 μg/ml DNase) and kept on ice. The cell suspension was kept on ice for a maximum of three hours, after which a new batch of cells was prepared.

### In Vivo Transplantation

Adult (9 weeks old) male (225-300 g) (n=30) NIH (NTac: NIH-Foxn1^{mu}) nude rats purchased from Taconic Biosciences A/S were grouped-housed in ventilated cages in a clean room under a 12-hr light/dark cycle with ad libitum access to sterile food and water. In addition to a standard rat diet, they were given peanuts to increase caloric intake.

The rats were anesthetized with isoflurane (5% for induction, 2-3% for maintenance), 1.2 L/min of O₂, and 0.6 L/min of atmospheric air, and placed in a stereotaxic frame (Stoelting) and unilaterally injected with 6-OHDA (Sigma-Aldrich A/S) (2 μl of 7 μg/μl free base in saline containing 0.02% ascorbic acid) (Tentilier et al., 2016) into the right MFB (anteroposterior (AP), −4.4; mediolateral (ML)-1.1; dorsoventral (DV), −7.6; tooth bar, 3.3) using a Hamilton syringe with a glass cannula attached. Following injection, the cannula was left in place for 5 min before being slowly retracted. The incision was sutured, and the animals were injected with buprenorphine (0.36 mg/kg) as an analgesic. Once the animals were fully awake, they were placed back into their cages with wet food and 0.009 mg/ml Temgesic in water.

Lesioning efficiency was assessed 3 weeks postsurgery using the amphetamine-induced rotation test, and animals that exhibited >5 rotations/min were used for further experiments. The selected rats were divided into 3 groups with a similar average number of amphetamine-induced rotations: the 6-OHDA lesion (no transplantation) group (n=8), the H9 cell-transplanted group (n=9) and the 4X cell-transplanted group (n=9) (FIG. 13B-C). Four weeks after lesioning, the animals in the H9 cell-transplanted and 4X cell-transplanted groups were stereotaxically injected into the striatum (AP, +0.5; ML, −3; DV, −4.6/4.8) with 250,000 cells of the respective cell type in a volume of 2.5 μl using a protocol similar to the one described above. All three groups were sacrificed 22 weeks postlesioning (i.e., 18 weeks after transplantation). Two transplanted rats did not complete the study and were euthanized due to health issues: one in the 4X cell-transplanted group (week 8 posttransplantation) due to a broken tail and one in H9 cell-transplanted group due to hindlimb paralysis (week 17 posttransplantation).

### Amphetamine-Induced Rotation Test

An amphetamine-induced rotation test was performed as described previously (Björklund et al., 2019) one week prior to transplantation to assess the effects of the lesions and 8 and 18 weeks posttransplantation. The animals were intraperitoneally (i.p.) injected with 5 mg/kg D-amphetamine and connected to a rotameter (LE 902, PanLab, Harvard Apparatus) coupled to a LE 3806 Multicounter (PanLab, Harvard Apparatus). The number of body rotations over a period of 90 min was recorded.

The data are expressed as the net number of full body turns per minute, with ipsilateral rotations having a positive value and contralateral rotations having a negative value. Animals exhibiting >5 turns/min were considered successfully lesioned. One rat had a technical issue during one of the rotation tests and was excluded from this behavioral test.

### Cylinder Test

The cylinder test was used to assess paw use asymmetry three weeks postlesioning (one week prior to transplantation) and 18 weeks posttransplantation. The animals were placed in a transparent Plexiglas cylinder (height of 30 cm, diameter of 20 cm), and two mirrors were placed behind the cylinder so that the cylinder surface could be fully visualized.

Spontaneous activity was video recorded for a total of 5 min. Data analysis was performed by a researcher blinded to the groups using VCL Media Player software in slow motion as previously described (Schallert et al., 2000). Because most of the exploratory motor activity of the animals was limited to the first 2 min and there was little movement after this timepoint, activity in the first 2 min were analyzed, and activity after this time point was analyzed only if the animal exhibited fewer than 10 movements (wall touches and rears).

The following behaviors were scored to determine the extent of forelimb-use asymmetry (Schallert et al., 2009): a) independent use of the left or right forelimb when touching the wall during a full rear or landing on the floor after a rear and b) simultaneous use of both the left and right forelimb to contact the wall of the cylinder during a full rear, for lateral movements along the wall (wall stepping) and for landing on the floor following a rear. The data are presented as the percentage of time each forelimb (left or right) or both forelimbs were used relative to all movements (wall and floor).

### Immunohistochemical Analysis of Brain Slices

The rats were killed 23 weeks after 6-OHDA-induced lesioning by an overdose of pentobarbital (50 mg/kg i.p.). During respiratory arrest, they were perfused through the ascending aorta with ice-cold saline followed by 4% cold PFA (in 0.1 M NaPB, pH 7.4). The brains were extracted, postfixed in PFA for 2 hours and transferred to 25% sucrose solution (in 0.02 M NaPB) overnight. The brains were sectioned into 35 μm thick coronal sections on a freezing microtome (Microm HM 450, Brock and Michelsen), separated into serial coronal sections (series of 8 for the striatum and the substantia nigra), and stored at −20° C.

Immunohistochemical staining was performed on free-floating brain sections using the following primary antibodies: mouse anti-rat TH (1:4000, MAB318, Merck Millipore), rabbit anti-Girk2 (1:500, APC-006, Alomone), rabbit anti-TH (1:1000, PelFreeze), mouse IgG1 anti-CALB1 (1:5000, 28k, SWANT), mouse IgG1 anti-HNA (1:200, 151181, Abcam), goat anti-FOXA2 (1:200, AF2400), sheep anti-hCOL1A1 (1:200, R&D Systems), rabbit anti-hCOL3A1 (1:1000), rabbit anti-EN1 (1:50), and rabbit anti-LMX1A (1:5000).

Immunohistochemistry was performed as previously described (Tentilier et al, 2016) with avidin-biotin-peroxidase complex (ABS Elite, Vector Laboratories) and 3,3-diaminobenzidine (DAB) as a chromogen to visualize the signal. The sections were mounted on chrome-alum gelatin-coated slides, dehydrated, and coverslipped. The slides were analyzed using a Olympus VS120 Slide Scanner (upright widefield fluorescence) with a 20× objective.

For immunofluorescence, free-floating sections were blocked in 5% normal donkey serum in 0.25% Triton X-100 in KBPS and then incubated overnight with the selected primary antibody in 2.5% donkey serum and 0.25% Triton X-100 in KPBS at room temperature. The sections were washed with KPBS, preblocked for 10 min in 1% donkey serum and 0.25% Triton X-100 in KPBS and incubated for 2 hours with the following species-specific fluorochrome-conjugated secondary antibodies made in donkey: Alexa Fluor 488-conjugated anti-mouse IgG (1:200, Jackson ImmunoResearch), Alexa Fluor 568-conjugated anti-goat IgG (1:1000, A11057, Invitrogen), Alexa Fluor 647-conjugated anti-rabbit IgG (1:200, Jackson ImmunoResearch), Alexa Fluor 568-conjugated anti-rabbit IgG (1:1000, A10042, Invitrogen), Alexa Fluor 647-conjugated anti-mouse IgG (1:1000, A-31571, Invitrogen) and Alexa Fluor 568-conjugated anti-mouse IgG1 (1:1000, A10037, Invitrogen), Alexa Fluor 568-conjugated anti-sheep IgG (1:1000, Invitrogen). DAPI (1:2000, Sigma-Aldrich A/S) was used for nuclear staining. The sections were mounted on chrome-alum gelatin-coated slides with Dako fluorescent mounting medium.

### Microscopic Analysis (TH-Positive Cell Number and Yield and Graft Volume)

Coronal sections (1:8) from each animal were immunostained for TH, and DA neurons in the graft were analyzed. An Olympus VS120 Slide Scanner (upright widefield fluorescence) was used to acquire images of the slides using a 20× objective. All sections with visible grafts were selected: 3-5 sections per H9 cell-transplanted animal and 4-8 sections per 4X cell-transplanted animal. The area in which the number of TH-positive cells was quantified included the striatum and globus pallidus, but TH-positive cells in the cortex and corpus callosum were not included. The images were analyzed by identifying cells in the region of interest (ROI) using QuPath software (Bankhead et al., 2017). The settings adapted for each section depending on the staining, and the following settings were used: detection image=optical density sum, requested pixel size=0.5 μm, background radius=15-30 μm, threshold=0.15-0.3, median filter radius=0-3 μm, sigma=0.7-2 μm, minimum area=85-130 μm2, maximum area=500-1200 μm2, max background intensity=2, cell expansion=2 μm. The cells were classified by shape, including that of the cell nucleus, and that the boundaries were smoothed.

To estimate the number of cells in a full graft, the total number of TH-positive cells per animal was determined with QuPath software and multiplied by 8, and the Abercrombie method (Abercrombie, 1946) was used to correct for double counting of cells spanning more than one section. The Abercrombie factor of each group was calculated as the average thickness per section divided by (the averaged thickness+the average TH-positive cell size). These numbers were calculated by sampling 3 sections and 18 cells per animal from 3 different animals per group. The total number of cells in a graft was calculated as the Abercrombie factor x the total number of TH-positive cells×8. The number of surviving cells (yield) was estimated per 100,000 transplanted cells. The volume of each graft was estimated as V=A1T1+A2T1+ . . . +AₙT1, where V is estimated volume, T1 is the sampling interval of a 1/8 series (8×35 μm), and A(n) is the area TH-positive area in the section (n) (Piao et al., 2021).

### Statistical Analysis

All statistical analyses were performed using GraphPad Prism v 9.1.1.225. One-way ANOVA or two-way ANOVA was performed, and Sidak's test was used for post hoc analysis when appropriate. Unpaired, two-tailed t-tests were used when comparing only the grafted groups. All data are presented as the mean±standard error of the mean (SEM) or ±standard deviation (SD) (as indicated). P<0.05 was considered significant.

### Example 2-Deletion of GBX2 and CDX1/2/4 Results in Increased Midbrain Identity

### Aim of Study

To determine if deletion of GBX2 and CDX1/2/4 results in increased midbrain identity.

### Materials and Methods

See example 1.

### Results

To enhance the differentiation of hESCs into dopaminergic neurons, we focused on knocking out transcription factors that regulate cell fate along the A-P axis. Initially, we investigated if knocking out GBX2^{-/-} in our hESCs (H9 cell line) would result in an increase in midbrain cell types when differentiated under conditions known to produce hindbrain and spinal cord cell types. We generated a GBX2^{-/-} human embryonic stem cell line. In the undifferentiated state, the cell line was transcriptionally indistinguishable from control hESCs and was capable of differentiating into neural progenitors.

### Gbx2^{-/-}

Using our previously published protocol that produces caudal neural progenitors (CNPs; (Denham et al., 2015)), we differentiated GBX2^{-/-} cells for four days to CNPs and compared them to hESC-derived CNPs (FIG. 1A). Immunostaining of H9 CNPs at day 4 showed no expression of OTX2 and positive staining for CDX2. GBX2−/− CNPs at day 4 showed few positive cells for OTX2 and positive staining for CDX2. 4xKO CNPs have a few cells positive for OTX2 and no cells are positive for CDX2 (cell pictures not shown). Indeed, when differentiated in the presence of a GSK3B inhibitor (GSK3i; CHIR99021) at a concentration known to give rise to the hindbrain and spinal cord (3 μM), the GBX2^{-/-} line showed a small but significant increase in the forebrain/midbrain marker OTX2 (FIG. 1B) and a significant reduction in CDX2 transcripts compared to H9 (FIG. 1C). Despite the increase in OTX2, the GBX2^{-/-} CNPs still expressed HOX genes related to the hindbrain and spinal cord cell types (FIG. 1D). Furthermore, by FACS analysis, we only observed a small increase in the number of OTX2 positive cell and a small decrease in the percentage of CDX2 positive cells (data not shown). GBX2^{-/-} CDX1, 2, 4^{-/-}

Based on the results from the GBX2^{-/-} line, we attempted to restrict further the potential of the cells along the A-P axis by knocking out the CDX gene family. Cdx2 is an upstream regulator of posterior Hox genes and is a key determinant of the spinal cord (Skromne et al., 2007). To ensure no expression or compensation from other CDX family members, we generated homozygous knockouts for all three CDX family members CDX1/2/4 by targeting their DNA binding domains using CRISPR (FIG. 1E). The resulting hESC line GBX2/−CDX1, 2, 4^{-/-} (hereafter called 4xKO) were differentiated for four days using the same CNP protocol (FIG. 1A). As expected, in the 4xKO CNPs, we could not detect CDX2 transcripts or CDX2 positive cells (FIG. 1C). Strikingly, the 4xKO day-4 neural progenitors showed a significant increase in OTX2 transcripts compared to H9 and GBX2^{-/-} derived CNPs (FIG. 1B).

To further understand the effects on gene expression, we performed RNA sequencing on the CNPs from all of the three cell lines (H9, GBX2^{-/-} and 4xKO). When we examined the HOX gene profile the 4xKO CNPs had a massive restriction in the expression of caudal HOX genes and did not express HOXA4 and HOX genes more posteriorly to HOXA13 (FIG. 1D). These results indicate that the 4xKO cell line was unable to generate progenitor cell types relating to rhombomere R4 and caudal to that.

We next wanted to know to what extent there were changes in the anterior genes. Firstly, we looked at the expression of forebrain genes in 4xKO CNPs and saw that there was no significant change in the expression of SIX3, DLX2 and FOXG1. However, transcripts for the forebrain/midbrain gene OTX2 were significant increase compared to H9 and GBX2^{-/-}. The midbrain genes PAX2, PAX5, EN1/2, and were also significantly increased compared to H9 and GBX2^{-/-} (FIG. 1C). Interestingly in GBX2^{-/-} cells we saw a reduction in anterior hindbrain genes such as EGR2 and MAFB, however, in 4xKO these genes increased in concordance with the loss in posterior HOX genes. Overall, the 4xKO cell line showed a posterior limit equivalent to rhombomeres R4 and significantly higher expression of midbrain and anterior hindbrain genes (FIG. 1C).

To explore further the potential of the 4xKO line, we differentiated the 4xKO cells and GBX2^{-/-} lines to day-11 CNPs under ventralising conditions (FIG. 2Q). We found that similar to the day-4 time point there were more transcripts for OTX2 in the 4xKO line compared to H9 or GBX2^{-/-} (FIG. 2A). Furthermore, we saw that the midbrain genes PAX2, PAX5, PAX8 were all up-regulated in 4xKO compared to H9 and GBX2^{-/-} (FIG. 2B, C, E). EN1, which in development spans the caudal midbrain and rhombomere R1, was also significantly up-regulated in 4xKO compared to H9 and GBX2 (p<0.01 and p<0.05 respectively; FIG. 2D). For the hindbrain gene KROX20, which is expressed in rhombomeres R3 and R5, it was significantly up-regulated (p<0.01; FIG. 2J), and MAFB, a marker of rhombomeres R5 and R6, was not significantly changed (FIG. 2K). IRX3 was still present but had significantly lower transcripts in the 4xKO compared to H9 and GBX2^{-/-} (FIG. 2F). These results showed that the HOX profile was again restricted in the 4xKO to a posterior limit up to and including HOXA3, however transcripts for HOXA3 were low and more posterior HOX genes undetected (FIG. 2L, M, N, O, P). Interestingly, the anterior HOX genes HOXA2 (FIG. 2G) maintained its expression in the 4xKO day-11 CNPs and HOXB2 (FIG. 2H) and HOXB1 (FIG. 2I) were significantly up-regulated compared to H9. These results show that there was a shift in the population anteriorly, whereby the cells preferentially adopted a midbrain or anterior hindbrain identity.

Immunofluorescent staining confirmed the change in population towards a midbrain identity. We identified the presence of OTX2⁺/EN1⁺ positive caudal midbrain progenitors in the day 11 4xKO cells but were undetected in the H9 control cells (data not shown).

These results indicate that under differentiation conditions known to generate CNPs, the 4xKO line could not produce spinal cord fates and had a restricted HOX profile up to rhombomere R4. Furthermore, the distribution of cell types was shifted anteriorly demonstrated by the presence of OTX2+/EN1+ cells, which were not detected in the control cell line under CNPs differentiation conditions.

### Conclusion

The provided data show that by knocking out (generating a GBX2^{-/-} cell line) alone or the four genes GBX2, CDX1, CDX2 and CDX4 (generating a GBX2^{-/-} CDX1, 2, 4^{-/-} cell line) cell lines with restricted differentiation potential were generated enabling them to more efficiently generate mesencephalic dopaminergic neuron progenitors or mesencephalic dopaminergic neurons.

### Example 3-Lineage-Restricted PSCs Efficiently Generate Caudal Midbrain Progenitors

### Aim of Study

To determine if the 4xKO line could more efficiently generate mesDA neurons when differentiated using a dopaminergic neuron protocol.

### Materials and Methods

See also examples 1 and 2.

### Results

We compared H9 to 4xKO and differentiated them using one of the most recent mesDA protocols (FIG. 3A) (Nolbrant et al., 2017). We first started by titrating the concentration of the GSK3i from 0.5 μM up to 1 μM to determine the optimal concentration for generating mesDA progenitors with H9. H9 and 4xKO cells were differentiated to day 16 (cell figures not shown).

In our lab, the optimal concentration for H9 was identified to be 0.6 μM, and at this concentration, the level of OTX2 transcripts was at the maximum at 0.6 μM and decreased at higher concentrations of GSK3i when assessed at day 16 (FIG. 3B). Furthermore, the transcript level for HOXA2 was at its lowest point between 0.5 and 0.6 μM of GSK3i (FIG. 3G). The concentration of the caudal midbrain marker CNPY1 was the highest between 0.5 μM and 0.6 μM, which significantly dropped as the concentration reached 1 μM (FIG. 3F). EN1 was also significantly higher at 0.5-0.6 μM and dropped significantly from 0.7 μM and 1 μM (FIG. 3E). These results were consistent with previous reports, which indicate that concentrations below 1 μM of the GSK3i are required for midbrain specification and concentrations approaching 1 μM or higher result in a dramatic shift to a hindbrain identity (Kirkeby et al., 2012).

When we compared the 4xKO cells across the same GSK3i concentrations, we saw a striking difference to H9. The 4xKO line had significantly higher transcripts for OTX2 and EN1 and LMX1A across all of the concentrations from 0.5 μM up to and including 1 μM compared to H9 (FIGS. 3B, 3E and 3C). The caudal midbrain marker CNPY1 was significantly higher from 0.65 μM up to and including 1 μM compared to H9 (FIG. 3F). These results indicated that the restriction in the potential of the 4xKO line results in the generate midbrain progenitors across a broader concentration of GSK3i concentration. Furthermore, the 4xKO line could also generate significantly more midbrain transcripts than the H9 can at the optimal concentration (cell figures not shown).

To explore further, why 4xKO are more efficient than H9 even under optimal conditions, we examined the range of cell types along the A-P axis that control cell types produced even under the most optimal conditions. We found that the control cells still expressed transcripts for HOXA2, which corresponded to the hindbrain (FIG. 3G), indicating that even with an optimized protocol the H9 line produced a large distribution of cell types along the A-P axis including hindbrain cell types. These results were in accordance with previous publications, which show expression of hindbrain cell types in cell lines even at optimal concentrations (Kirkeby et al., 2012). However, the 4xKO cells expressed a significantly lower concentration of HOXA2 compared to H9. Furthermore, the level of HOXA2 in the H9 increased significantly when higher concentrations of GSK3i were used, whereas the level of HOXA2 remained unchanged in the 4xKO line across the concentrations (FIG. 3G).

Immunofluorescent staining confirmed the above results and showed that there was an abundance of EN1 positive cells at 0.6 μM and up to 1 μM of GSK3i concentrations in the 4xKO line (data not shown). However, in the H9 line EN1 positive cells were confined to a restricted concentration of 0.6 μM, which indicated that a broader distribution of cells along the A-P axis is produced in H9 cells.

### Conclusion

These results showed that the 4xKO could not produce spinal cord cell types and confined the majority of cells to a midbrain and anterior hindbrain identity resulting in a higher proportion of mesDA progenitors even at suboptimal concentrations of GSK3i.

### Example 4-Lineage-Restricted PSCs Differentiated Under Hindbrain Conditions Generate Midbrain Progenitors and mesDA Neurons

### Aim of Study

To determine the cell types that were produced in the 4xKO line under conditions that normally give rise to predominantly hindbrain cell types.

### Materials and Methods

See also examples 1-3.

### Results

We compared H9 and 4xKO cells at 1 μM concentration of the GSK3i inhibitor by single-cell analysis at day 16. As expected, we identified that in both conditions, a significant proportion of cells were ventralised to the floor plate (H9 48%, 4xKO 73%). However, we saw a significant variation in the distribution of cells along the A-P axis between the two cell lines (p<0.00001; FIG. 4). There were significantly more cells that expressed the anterior gene OTX2 in the 4xKO cells (H9 1%; 4xKO 43%). Furthermore, the 4xKO condition were massively enriched in caudal midbrain cells (OTX2+/En1+; H9 0%; 4xKO 35%). Cells corresponding to rhombomere R1 (Otx2−/En1+) were also enriched in the 4xKO cells (H9 3%; 4xKO 39%). Hindbrain and more caudal cell types denoted by the expression of HOX genes were significantly higher in H9 compared to the 4xKO cells (H9 85%; 4xKO 2%), indicating that the majority of cells in H9 corresponded to a hindbrain identity, whereas the distribution of 4xKO cells corresponded to the caudal midbrain and anterior hindbrain (FIG. 4).

We next wanted to know if the 4xKO cell line could produce mesDA neurons under these caudalising conditions. By single cell analysis, we found that at 1 μM of GSK3i we were able to produce approximately 11.6% of dopaminergic neurons in the 4xKO line, compared to 2.8% in the control. Immunofluorescent analysis of 100 day organoids from H9 and 4xKO also showed that the majority of Dopaminergic neurons also expressed the ventral marker FOXA2, indicating that we were successfully generating mesDA neurons. 4xKO organoids contain Dopaminergic neurons TH and FOXA2 positive. No cells double positive for FOXA2 and TH could be detected in the H9 organoids (data not shown). Midbrain organoids at day 100 differentiated from 4xKO and H9 using 1 μM of GSK3i, 4xKO organoids contain Dopaminergic neurons TH and FOXA2 positive. No cells double positive for FOXA2 and TH could be detected in the H9 organoids.

### Conclusion

These data further substantiate that starting from a GBX2^{-/-} CDX1, 2, 4^{-/-} cell line, it is possible to more efficiently generate mesDA neurons compared to a control cell line.

### Example 5-Knockout of Anterior HOX Genes Further Restricts the Potential of PSCs and Increases Midbrain Cells Types-8xKO

### Aim of Study

To determine if Knockout of anterior HOX genes would further restrict the potential of PSCs and increases midbrain cells types.

### Materials and Methods

See also examples 1-4.

### Results

Based on the single-cell data, differentiation of 4xKO cell into mesDA progenitors produced only 1% of cells expressing HOX genes. However, under high caudalising conditions, the cell line could still express hindbrain HOX genes (FIG. 1C). The CDX gene family does not regulate anterior HOX genes in rhombomeres R2-R4. We therefore selected HOXA1/2 and HOXB1/2 and GBX1 as additional genes to knockout. HOXA1/2 and HOXB1 and GBX1 were successfully targeted (FIG. 1F). Despite this, we continued with the new combined 4xKO and HOX knockout cell line (hereafter referred to as 8xKO) and differentiated them to day-4 CNPs (FIG. 5A). The 8xKO cells at day four had significantly increased transcripts levels for OTX2, which was supported by the massive increase (30%) in OTX2 positive cells compared to 2% in the 4xKO cells (FIG. 5B-C). Further analysis by RNA sequencing also showed that there was a significant increase in the expression of midbrain gene compared to the 4xKO line (FIG. 5D-E).

### Conclusion

Further knocking out HOXA1/2, HOXB1 and GBX1 thereby generating a cell line being GBX2^{-/-} CDX1, 2, 4^{-/-} HOXA1,2^{-/-} HOXB1^{-/-} and GBX1^{-/-}, results in a significant increase in the expression of midbrain genes compared to the 4xKO line.

### Example 6-Genes that Represent Cell Types Along the Dorsal Ventral Axis

### Aim of Study

To evaluate the impact of the expression of genes that represent cell types along the dorsal ventral axis.

### Materials and Method

See previous examples.

### Results

Analysis of the 4xKO cell line (knockout of GBX2, CDX1, CDX2 and CDX4) showed that the specification of cells along the anterior posterior axis has been disrupted and resulted in the cells producing more midbrain cell types. When we analyzed the expression of genes that represent cell types along the dorsal ventral axis the control and 4xKO cell line show no major changes in the percentage of cells that are generated (FIG. 3D). In particular, we saw that both cell lines are capable of generating basal plate cells, indicated by the expression of NKX gene family members and PHOX2A/B (FIG. 6A).

Single-cell analysis at day 28 of dopaminergic differentiation, in 4X and H9 cells combined, identified 15 cell clusters. The anterior ventral marker NKX2.1 was prominently expressed in clusters 11 and 6, and the medial marker for the dorsal ventral axis PAX6 was expressed in cluster 12 (FIG. 6B).

### Conclusion

Mesencephalic dopaminergic neurons arise from the floor plate, the most ventral population in the neural tube. NKX family members are important in specifying the lateral (basal plate) populations of the neural tube.

Based on the presented data, it is concluded that the same knockout approach can be extended to the NKX family members and other basal plate markers to prevent those cell types and increase the specification to a mesencephalic dopaminergic neural progenitor.

### Example 7-LR-PSCs Efficiently Generate mesDA Neurons Under Conditions that Favor a Hindbrain Identity

### Aim of Study

To examine the extent to which midbrain floor plate progenitors can produce mesDA neurons.

### Materials and Method

See previous examples.

### Results

The 1 μM GSK3i protocol was extended to 62 DIV. At 62 DIV, the two cell lines occupied almost completely separate clusters (Chi-square, P<0.0001) (data not shown). 4X cells were broadly divided into two main cell types: hindbrain r1 floor plate clusters expressing FOXA2, SHH, NETRIN1, SPON1 and EN1 (clusters 4, 5 and 6) and neuronal clusters (clusters 0 and 8; FIG. 11A). The neuronal clusters contained mesDA neurons identified by the expression of TH, FOXA2, LMX1A and EN1 (FIGS. 11A-B). Clusters 0 and 8 was comprised almost entirely of 4X cells (4X: 82% and 77%, H9: 18% and 23%) (data not shown). Upon closer inspection of the difference between clusters 0 and 8, we identified a subset of cells within cluster 8 that expressed NKX2.1, a marker of hypothalamic neurons.

In contrast to 4X cells, H9 cells formed one main connected set of clusters (clusters 1, 2, 3, and 7) and two small isolated clusters (clusters 9 and 10) (data not shown). All six clusters were dominated by cells expressing markers indicative of vascular leptomeningeal cells (VLMCs), i.e., COL3A1, IFITM2 and S100A11 (FIG. 11A). Interestingly, EN1 was largely absent from the VLMC clusters (data not shown). Cluster 7 also contained a population (41%) of cells expressing STMN2, SEMA3C and PDLIM1 (FIGS. 11A and 11C), which, according to a single-cell brain atlas, corresponded to a subtype of peripheral sensory neurons.

We next wanted to examine the subtypes of mesDA neurons by first subclustering of TH-positive neurons (data not shown). To distinguish between substantia nigra and ventral tegmental area (VTA) DA neurons, we assessed the expression of GIRK2 (also known as KCNJ6) and CalbindinD (CALB1). GIRK2 was highly expressed in the subclusters 1 and 3, and accounted for 38% of the TH population, and only a small proportion of TH neurons expressed CALB1 (13.6%; FIG. 11D).

To further support our single-cell sequencing results, histological analysis was performed. We used the same growth factor paradigm but adapted a differentiation protocol to generate organoids to provide an optimal environment for the survival of neurons (FIG. 12A). At 83 DIV, we observed a large population of FOXA2/TH double-positive DA neurons within organoids produced from 4X cells (data not shown). In contrast, TH-positive cells were occasionally scattered throughout organoids produced by H9 cells; however, we rarely detected TH-positive cells coexpressing FOXA2 (data not shown). These results were consistent with the histological analysis of our 2D cultures at 62 DIV (data not shown). Further examination of TH-positive neurons showed that, in accordance with our single-cell data, the most abundant population of TH neurons derived from 4X cells coexpressed GIRK2 (data not shown) and that there was a small population of CALB1/TH double-positive neurons (data not shown).

According to the single-cell sequencing data, the majority of H9 cells were VLMCs (Clusters 1, 2, 3, 7, 9, 10; 93% of H9 cell). To confirm this finding, we examined the expression of VLMC markers in organoids at 83 DIV, and we identified a large population of COL3A1/COL1A1 double-positive cells with a nonneuronal morphology among H9 cells (data not shown). No cells positive for COL3A1 or COL1A1 were identified among 4X cells (data not shown).

### Conclusion

mesDA neurons can be generated from 4x cells under caudalizing conditions.

### Example 8-Genes that Represent Cell Types Along the Dorsal Ventral Axis. DA Neurons Derived from LR-PSCs Exhibit Pacemaker Activity

### Aim of Study

To examine the electrophysiological properties of the DA neurons.

### Materials and Method

See previous examples.

### Results

We performed in vitro electrophysiological recordings in whole-cell patch-clamp configuration between DIV 80 and DIV 84 (data not shown). We observed that the cells developed into electrophysiologically mature neurons, as measured by their ability to generate repetitive action potentials upon somatic current injection (FIG. 12B). Recordings in current-clamp mode revealed spontaneous pacemaker activity characteristic of a DA neuron identity, with a mix of single spikes and phasic bursts (FIG. 12C). Membrane oscillations collapsed at potentials below −50 mV (data not shown). The firing frequency in our sample ranged from 1 to 5 Hz (FIG. 12D). Furthermore, HPLC analysis of cell extracts showed that the DA content in the 4X cells was significantly higher than that in the H9 cells (287.4 nmol/g in 4X cells vs. 65.1 nmol/g in H9 cells, P=0.002; FIG. 12E).

### Conclusion

Based on the present data, it is concluded that the dopaminergic neurons prepared from the 4X cells are capable of developing into electrophysiologically mature neurons.

### Example 9-Genes that Represent Cell Types Along the Dorsal Ventral Axis. Analysis of 4X Cells In Vivo in a Parkinson's Disease Rat Model

### Aim of Study

When using current DA neuron differentiation protocols, DA neurons account for only a small percentage of cells of the entire graft when mesDA progenitors are grafted in vivo. We investigated how our 4X LR-PSCs behave in vivo when transplanted into a rodent model of Parkinson's disease.

### Materials and Method

See previous examples.

### Results

To confirm the results of our single-cell analysis, we used the same midbrain differentiation protocol with an unfavorable caudalizing concentration of GSK3i (1 μM).

A total of 250,000 4X cells or H9 cells were transplanted into the striata of nude rats with 6-OHDA-induced medial forebrain bundle (MFB) lesions 4 weeks after lesioning. A third group of lesioned rats that did not undergo transplantation was used as a lesion control (6-OHDA, see FIG. 13A for study design). At the time of transplantation, all three groups of rats exhibited a similar number of amphetamine-induced ipsilateral rotations/min (limit for inclusion: 5 rotations/min, FIG. 13B), confirming significant loss of DA striatal innervation. All three groups showed forelimb asymmetry in the cylinder test, with the rats using mostly the ipsilateral forepaw (6-OHDA 52.4%; H9 70.4% and 4X67.4% of total) and almost never the contralateral forepaw (6-OHDA 1.3%; H9 1.3% and 4X 0% of total) to touch the walls or land on the floor after rearing, further supporting the induction of a DA deficit by 6-OHDA (FIG. 13C).

Eight weeks posttransplantation, rats that received 4X cells showed complete correction of amphetamine-induced ipsilateral rotation (pretransplant: 10.6 vs 8w: 0.35 rotations/min), suggesting that a sufficient amount of dopamine was released in the striatum to normalize (FIG. 13D) or even overcompensate for this behavior, as suggested by the number of contralateral rotations (−3.12 rotations/min) observed 18 weeks posttransplantation. However, H9 cells-transplanted rats presented a statistically similar number of ipsilateral rotations as the control 6-OHDA lesion group (pretransplant: 9.8; 8w: 9.7 and 18w: 9. 2 rotations/min) throughout the entire experiment, showing only a significant reduction in the number of rotations compared to pretransplantation values at 18 weeks (H9 pretransplant: 11.5; 8w: 12.6 and 18w: 5.6 rotations/min, FIG. 13D). Analysis of spontaneous motor behavior in the cylinder test confirmed the significant improvement in 4X cell-transplanted rats, as these rats used the contralateral forelimb alone (9.7% of total) or together with the ipsilateral forelimb (both 46.3%) during the test at week 18 (FIG. 13E). However, both H9 cell-transplanted and 6-OHDA lesion rats used mostly the ipsilateral forelimb (76.1% and 79.3% of total respectively), using both forelimbs less than 30% of the time but almost never using the contralateral impaired forelimb when rearing in the cylinder, as observed before transplantation (FIG. 13E). Therefore, 4X cell transplantation significantly improved both drug-induced and spontaneous motor behavior after 6-OHDA-induced lesioning of the MFB.

Postmortem histological analysis of the brains showed that rats transplanted with 4X cells had graft-derived TH-positive cells in the area of injection, i.e., the striatum, as well as in globus pallidus, the corpus callosum and the area of the cortex above the striatum (FIG. 13F). However, H9-derived TH-positive cells remained mostly in the striatum and were also found in the globus pallidus in few animals. Quantification of graft-derived TH-positive cells (in the striatum and globus pallidus) showed that there were significantly more TH-positive cells per graft in 4X cell-transplanted rats (an average of 23,520 TH-positive cells per graft) than H9 cell-transplanted rats (1,898 TH-positive cells per graft), resulting in a larger yield (9,408 TH-positive cells per 100,000 transplanted 4X cells vs. 759 TH-positive cells per 100,000 transplanted H9 cells) (FIGS. 13G-H). The TH-positive 4X cell graft extended across 6-8 coronal A-P striatal sections (in a series of 8), while the H9 cell graft occupied 4-5 sections. Thus, the estimated graft volume was 61% larger in the 4X cell-transplanted rats (20.46 mm³) than in the H9 cell-transplanted rats (12.69 mm³) (FIG. 13I). The increase in TH-positive cell number resulted in a significantly higher density of TH cells in the graft in the 4X cell-transplanted group (1,090±464 cells/mm³ vs. 143±49 cells/mm³ in the H9 cell-transplanted group; P<0.0001), which is in agreement with the rapid and robust behavioral recovery observed in the 4X cell-transplanted group.

Further examination of the graft showed that all TH-positive neurons identified within the 4X and H9 cell grafts coexpressed the human nuclear marker human nuclear antigen (HNA) (data not shown). In the grafts of 4X cell-transplanted rats, TH-positive neurons coexpressed FOXA2, LMX1A and EN1, indicating that they were mesDA neurons (data not shown). To distinguish between A9 and A10 neurons, we calculated the proportion of TH-positive neurons expressing GIRK2 and CALB1 and found that 75.4%+4.99 of TH-positive neurons were GIRK2-positive (FIG. 13J).

Interestingly, we also found that TH-positive neurons derived from H9 cells were positive for FOXA2, LMX1A and EN1 (data not shown). This was in contrast to our in vitro experiments, in which TH-positive neurons derived from H9 cells rarely expressed FOXA2 (data not shown), suggesting that the in vivo environment is more permissive for the development and survival of TH-positive neurons than an in vitro environment. Since the in vitro data showed that H9 cells produced a large number of VLMCs, we examined the expression of vascular markers in the grafts of both 4X cell-transplanted rats and H9 cell-transplanted rats. Within the H9 cell grafts, we identified a large population of COL3A1/COL1A1/HNA triple-positive cells, whereas in 4X cell grafts, we rarely detected COL1A1-positive cells coexpressing the marker HNA (data not shown).

### Conclusion

Overall, the in vivo histological data showed that 4X cells were capable of producing a robust population of mesDA neurons, consistent with the rapid motor recovery seen at 8 weeks.

### REFERENCES

Ang, S. L., Jin, O., Rhinn, M., Daigle, N., Stevenson, L., Rossant, J., 1996. A targeted mouse Otx2 mutation leads to severe defects in gastrulation and formation of axial mesoderm and to deletion of rostral brain. Development 122, 243-252.
Castelo-Branco, G., Wagner, J., Rodriguez, F. J., Kele, J., Sousa, K., Rawal, N., Pasolli, H. A., Fuchs, E., Kitajewski, J., Arenas, E., 2003. Differential regulation of midbrain dopaminergic neuron development by Wnt-1, Wnt-3a, and Wnt-5a. Proc. Natl. Acad. Sci. U.S.A 100, 12747-12752. https://doi.org/10.1073/pnas.1534900100
Denham, M., Hasegawa, K., Menheniott, T., Rollo, B., Zhang, D., Hough, S., Alshawaf, A., Febbraro, F., Ighaniyan, S., Leung, J., Elliott, D. A., Newgreen, D. F., Pera, M. F., Dottori, M., 2015. Multipotent caudal neural progenitors derived from human pluripotent stem cells that give rise to lineages of the central and peripheral nervous system. Stem Cells 33, 1759-1770. https://doi.org/10.1002/stem. 1991
Fasano, C. A., Chambers, S. M., Lee, G., Tomishima, M. J., Studer, L., 2010. Efficient Derivation of Functional Floor Plate Tissue from Human Embryonic Stem Cells. Cell Stem Cell 6, 336-347. https://doi.org/10.1016/j.stem.2010.03.001
Friling, S., Andersson, E., Thompson, L. H., Jonsson, M. E., Hebsgaard, J. B., Nanou, E., Alekseenko, Z., Marklund, U., Kjellander, S., Volakakis, N., Hovatta, O., El Manira, A., Björklund, A., Perlmann, T., Ericson, J., 2009. Efficient production of mesencephalic dopamine neurons by Lmx1a expression in embryonic stem cells. Proc. Natl. Acad. Sci. U.S.A 106, 7613-8. https://doi.org/10.1073/pnas.0902396106
Gill, K. P., Denham, M., 2020. Optimized Transgene Delivery Using Third-Generation Lentiviruses. Curr. Protoc. Mol. Biol. 133, 1-21. https://doi.org/10.1002/cpmb.125
Hynes, M., Porter, J. A., Chiang, C., Chang, D., Tessier-Lavigne, M., Beachy, P. A., Rosenthal, A., 1995. Induction of midbrain dopaminergic neurons by sonic hedgehog. Neuron 15, 35-44. https://doi.org/10.1016/0896-6273(95)90062-4
Kabadi, A. M., Ousterout, D. G., Hilton, I. B., Gersbach, C. A., 2014. Multiplex CRISPR/Cas9-based genome engineering from a single lentiviral vector. Nucleic Acids Res. 42. https://doi.org/10.1093/nar/gku749
Kirkeby, A., Grealish, S., Wolf, D. A., Nelander, J., Wood, J., Lundblad, M., Lindvall, O., Parmar, M., 2012. Generation of Regionally Specified Neural Progenitors and Functional Neurons from Human Embryonic Stem Cells under Defined Conditions. Cell Rep. 1, 703-714. https://doi.org/10.1016/j.celrep.2012.04.009
Kirkeby, A., Nolbrant, S., Tiklova, K., Heuer, A., Kee, N., Cardoso, T., Ottosson, D. R., Lelos, M. J., Rifes, P., Dunnett, S. B., Grealish, S., Perlmann, T., Parmar, M., 2017. Predictive Markers Guide Differentiation to Improve Graft Outcome in Clinical Translation of hESC-Based Therapy for Parkinson's Disease. Cell Stem Cell 20, 135-148. https://doi.org/10.1016/j.stem.2016.09.004
La Manno, G., Gyllborg, D., Codeluppi, S., Nishimura, K., Salto, C., Zeisel, A., Borm, L. E., Stott, S. R. W., Toledo, E. M., Villaescusa, J. C., Lönnerberg, P., Ryge, J., Barker, R. A., Arenas, E., Linnarsson, S., 2016. Molecular Diversity of Midbrain Development in Mouse, Human and Stem Cells. Cell (in press). https://doi.org/10.1016/j.cell.2016.09.027
Nolbrant, S., Heuer, A., Parmar, M., Kirkeby, A., 2017. Generation of high-purity human ventral midbrain dopaminergic progenitors for in vitro maturation and intracerebral transplantation. Nat. Protoc. 12, 1962-1979. https://doi.org/10.1038/nprot.2017.078
Rivetti, P., Romanov, R. A., Spigolon, G., Masini, D., Martín-montañez, E., Toledo, E. M., Manno, G. La, Feyder, M., Pifl, C., Ng, Y., Sánchez, S. P., Linnarsson, S., Wernig, M., Harkany, T., Fisone, G., Arenas, E., 2017. Induction of functional dopamine neurons from human astrocytes in vitro and mouse astrocytes in a Parkinson's disease model. Nat. Biotechnol. https://doi.org/10.1038/nbt.3835
Skromne, I., Thorsen, D., Hale, M., Prince, V. E., Ho, R. K., 2007. Repression of the hindbrain developmental program by Cdx factors is required for the specification of the vertebrate spinal cord. Development 134, 2147-2158. https://doi.org/10.1242/dev.002980
Corti et al. Direct reprogramming of human astrocytes into neural stem cells and neurons. Exp Cell Res. 2012 Aug. 1; 318 (13-16): 1528-1541.
Piao, J. et al. Preclinical Efficacy and Safety of a Human Embryonic Stem Cell-Derived Midbrain Dopamine Progenitor Product, MSK-DA01. Cell Stem Cell 28, 217-229.e7 (2021).
Bankhead, P. et al. QuPath: Open source software for digital pathology image analysis. Sci. Rep. 7, 16878 (2017).
Abercrombie, M. Estimation of nuclear population from microtome sections. Anat. Rec. 94, 239-247 (1946).
Tentillier, N. et al. Anti-Inflammatory Modulation of Microglia via CD163-Targeted Glucocorticoids Protects Dopaminergic Neurons in the 6-OHDA Parkinson's Disease Model. J. Neurosci. 36, 9375-9390 (2016).
Schallert, T., Fleming, S. M., Leasure, J. L., Tillerson, J. L. & Bland, S. T. CNS plasticity and assessment of forelimb sensorimotor outcome in unilateral rat models of stroke, cortical ablation, parkinsonism and spinal cord injury. Neuropharmacology 39, 777-787 (2000).
Björklund, A. & Dunnett, S. B. The Amphetamine Induced Rotation Test: A Re-Assessment of Its Use as a Tool to Monitor Motor Impairment and Functional Recovery in Rodent Models of Parkinson's Disease. J. Parkinsons. Dis. 9, 17-29 (2019).
Lunde, A. & Glover, J. C. A versatile toolbox for semi-automatic cell-by-cell object-based colocalization analysis. Sci. Rep. 10, 19027 (2020).
Metsalu, T. & Vilo, J. ClustVis: a web tool for visualizing clustering of multivariate data using Principal Component Analysis and heatmap. Nucleic Acids Res. 43, W566-W570 (2015).

1. A pluripotent stem cell, comprising an inactivated GBX2 gene.

2-27. (canceled)

28. The pluripotent stem cell according to claim 1, wherein GBX2, CDX1, CDX2 and CDX4 genes are inactivated.

29. The pluripotent stem cell according to claim 1, wherein GBX2, CDX1, CDX2, CDX4, GBX1, HOXA1, HOXA2 and HOXB1 genes are inactivated.

30. The pluripotent stem cell according to claim 1, wherein the GBX2 gene is inactivated by gene knock-out or introduction of a stop codon.

31. A method for cell lineage restricted cell differentiation of a pluripotent stem cell or for the generation of a dopaminergic neuron progenitor cell or a cell having lineage thereto comprising differentiating the pluripotent stem cell of claim 1 or generating a dopaminergic neuron progenitor cell or a cell having lineage thereto from the pluripotent stem cell of claim 1.

32. A dopaminergic neuron progenitor cell or a cell having lineage thereto, comprising an inactivated GBX2 gene.

33. The dopaminergic neuron progenitor cell or a cell having lineage thereto according to claim 32, wherein GBX2, CDX1, CDX2 and CDX4 genes are inactivated.

34. The dopaminergic neuron progenitor cell or a cell having lineage thereto according to claim 32, wherein GBX2, CDX1, CDX2, CDX4, GBX1, HOXA1, HOXA2 and HOXB1 genes are inactivated.

35. The dopaminergic neuron progenitor cell or a cell having lineage thereto according claim 32, wherein the dopaminergic neuron progenitor cell or a cell having lineage thereto:
expresses FOXA2, LMX1A, OTX2, EN1, and/or TH; and/or
expresses FOXA2, LMX1A, OTX2, EN1, SPRY1, WNT1, CNPY1, PAX8, ETV5, PAX5, SP5, and/or
is TLE4 positive.

36. The dopaminergic neuron progenitor cell or a cell having lineage thereto according to claim 32, wherein the dopaminergic neuron progenitor cell or a cell having lineage thereto is selected from the group consisting of a dopaminergic neuron cell, a dopaminergic neuron progenitor cell, a mesencephalic dopaminergic progenitor neuron cell, a mesencephalic dopaminergic neuron, a midbrain floor plate progenitor cell, and a caudal midbrain progenitor cell.

37. A method for treating or alleviating a neurodegenerative disorder in a subject, the method comprising grafting the dopaminergic neuron progenitor cell or a cell having lineage thereto according to claim 32, into the brain of the subject.

38. The method according to claim 37, wherein the neurodegenerative disorder is selected from the group consisting of Alzheimer's disease, Amyotrophic lateral sclerosis, Friedreich's ataxia, Huntington's disease, Lewy body disease, Parkinson's disease and Spinal muscular atrophy.

39. The method according to claim 37, wherein the neurodegenerative disorder is Parkinson's disease.

40. A method for making a pluripotent stem cell according to claim 1, the method comprising inactivating a GBX2 gene in a pluripotent stem cell.

41. The method according to claim 40, comprising inactivating GBX2, CDX1, CDX2 and CDX4 genes in the pluripotent stem cell.

42. The method according to claim 40, comprising inactivating GBX2, CDX1, CDX2, CDX4, GBX1, HOXA1, HOXA2 and HOXB1 genes in the pluripotent stem cell.

43. A method for making a dopaminergic neuron progenitor cell or a cell having lineage thereto the method comprising:
providing a pluripotent stem cell according to claim 1; and
differentiating the pluripotent stem cell into a dopaminergic neuron progenitor cell or a cell having lineage thereto.

44. The method of claim 31, wherein the cell having lineage to the dopaminergic neuron progenitor cell is selected from the group consisting of a dopaminergic neuron progenitor cell, a dopaminergic neuron cell, and a mesencephalic dopaminergic neuron.
